(19) United States
(12) Patent Application Publication
(10) Pub. No.: US 2020/0016150 A1
(43) Pub. Date: Jan. 16, 2020

(54) TREATMENT OF THE PRURITIC SYMPTOMS OF LIVER DISEASE

(71) Applicant: TREVI THERAPEUTICS, INC., New Haven, CT (US)

(72) Inventors: Thomas Sciascia, Belmont, MA (US); Amale Hawi, Ridgefield, CT (US)

(21) Appl. No.: 16/507,433

(22) Filed: Jul. 10, 2019

Related U.S. Application Data
(60) Provisional application No. 62/696,610, filed on Jul. 11, 2018.

(51) Int. Cl. A61K 31/485; A61P 1/16; A61K 9/00; A61K 45/06; A61K 9/20

(52) U.S. Cl. CPC A61K 31/485; A61P 1/16; A61K 9/0053; A61K 45/06; A61K 9/2009; A61K 9/2054; A61K 9/205; A61K 9/2013; A61K 9/2018

(57) ABSTRACT

The present disclosure relates to methods for treating patients with pruritus associated with liver disease with anti-pruritic compositions; methods for treating patients with pruritus associated with obstructive cholestasis secondary to bile duct obstruction due to non-hepatic tissue disease; and the anti-pruritic compositions used in such methods.

### CROSS-REFERENCE TO RELATED APPLICATIONS

The present application claims the benefit of priority to U.S. Provisional Application Ser. No. 62/696,610, filed Jul. 11, 2018, the contents of which are hereby incorporated by reference in their entirety.

### FIELD OF THE INVENTION

The present disclosure relates in some embodiments to methods for treating patients with pruritus associated with liver disease with anti-pruritic compositions (such as nalbuphine compositions); methods for treating patients with pruritus associated with liver disease with obstructive cholestasis secondary to bile duct obstruction due to non-hepatic tissue disease and the anti-pruritic compositions used in such methods.

### BACKGROUND

Pruritus, or itch, is a sensation that stimulates the desire to scratch. Pruritus can be either generalized to multiple non-contiguous anatomical areas or localized to one specific anatomical area over the body skin surface. The cause of pruritus is not fully understood. Proposed contributors to the pathogenesis of pruritus may include anemia or other manifestation of erythropoietin deficiency, histamine release from skin mast cells, skin dryness, secondary hyperparathyroidism, hyperphosphatemia with increased calcium phosphate deposition in the skin and alterations in the endogenous opioidergic system with overexpression of opioid μ-receptors.

Pruritus is a common symptom of chronic liver disease. As with other pruritic conditions (above), the etiology of pruritus associated with liver disease (or liver itch) is not fully understood. However, liver itch is often refractory to treatment with common anti-pruritic agents, and there is a need for effective treatments of the condition.

### SUMMARY OF THE INVENTION

The present disclosure, among other things, provides methods of treating pruritus comprising administering an effective amount of an anti-pruritus agent to a patient in need of such treatment. In some embodiments, the anti-pruritus agent is nalbuphine or a pharmaceutically acceptable salt, solvate or ester thereof.

In some embodiments, the patient in need of a treatment of pruritus is a patient with pruritus associated with liver disease. In some embodiments, the patient has chronic pruritus associated with liver disease. In some embodiments, the patient in need of a treatment of pruritus is a patient with pruritus associated with obstructive cholestasis secondary to bile duct obstruction due to non-hepatic tissue disease.

In some embodiments, the patient in need of a treatment of pruritus is a patient with pruritus associated with liver disease that is refractory to other therapies. In some embodiments, the patient's pruritus associated with liver disease is refractory to treatment with other anti-pruritus agents. In some embodiments, the patient's pruritus associated with liver disease (“liver itch”) is refractory to treatment with bile sequestrants. In some embodiments, the patient's pruritus associated with liver disease is refractory to treatment with rifampicin. In some embodiments, the patient's pruritus associated with liver disease is refractory to treatment with μ-opioid antagonists. In some embodiments, the patient's pruritus associated with liver disease is refractory to treatment with κ-opioid agonists.

In some embodiments, the patient in need of a treatment of pruritus is a patient with pruritus associated with a liver disease selected from cholestatic liver disease (e.g., primary biliary cholangitis and primary sclerosing cholangitis), infectious hepatitis; cirrhotic liver disease, drug-induced liver disease, idiopathic portal hypertension, congenital malformations or genetic diseases affecting liver function, sarcoidosis, primary or metastatic neoplasm involvement of the liver and autoimmune hepatitis-cholangitis (Overlap syndrome).

According to some embodiments of the present disclosure, the method of treating pruritus associated with liver disease comprises administering for at least a week to a patient in need thereof a daily dose of at least about 15 mg nalbuphine or a pharmaceutically acceptable salt, solvate or ester thereof. In some embodiments, the method of treating pruritus associated with liver disease comprises administering for at least a week to a patient in need thereof at least about 30 mg nalbuphine or a pharmaceutically acceptable salt, solvate or ester thereof. In some embodiments, the method of treating pruritus associated with liver disease comprises administering for at least a week to a patient in need thereof at least about 60 mg nalbuphine or a pharmaceutically acceptable salt, solvate or ester thereof. In some embodiments, the method of treating pruritus associated with liver disease comprises administering for at least a week to a patient in need thereof a daily dose of at least about 120 mg nalbuphine or a pharmaceutically acceptable salt, solvate or ester thereof. In some embodiments, the method of treating pruritus associated with liver disease comprises administering for at least a week to a patient in need thereof a daily dose of at least about 180 mg of nalbuphine or a pharmaceutically acceptable salt, solvate or ester thereof. In some embodiments, the method of treating pruritus associated with liver disease comprises administering for at least a week to a patient in need thereof a daily dose of at least about 360 mg of nalbuphine or a pharmaceutically acceptable salt, solvate or ester thereof. In some embodiments, about 15 mg of the anti-pruritus agent is administered twice a day. In some embodiments, about 30 mg of the anti-pruritus agent is administered twice a day. In some embodiments, about 60 mg of the anti-pruritus agent is administered twice a day. In some embodiments, about 90 mg of the anti-pruritus agent is administered twice a day. In some embodiments, about 180 mg of the anti-pruritus agent is administered once a day. In some embodiments, about 180 mg of the anti-pruritus agent is administered twice a day. In some embodiments, about 360 mg of the anti-pruritus agent is administered once a day.

In some embodiments, the anti-pruritus agent is administered for about 8 weeks. In some embodiments, the anti-pruritus agent is administered for about 10 weeks. In some embodiments, the anti-pruritus agent is administered for about 12 weeks. In some embodiments, the anti-pruritus agent is administered for about 18 weeks. In some embodiments, the anti-pruritus agent is administered for about 50 weeks.

In some embodiments, after the treatment the patient experiences a substantial reduction in itch compared to prior to the treatment.

In some embodiments, the method of treating pruritus further includes a step of titrating the dose of the anti-pruritus agent for at least about one week until a steady state is achieved in the patient. In one embodiment, the titration is conducted for about 2 weeks until a steady state is achieved in the patient. In another embodiment, the titration is conducted for about 7 days to about 30 days until a steady state is achieved in the patient. In another embodiment, the titration is conducted for about 12 days to about 20 days until a steady state is achieved in the patient.

In some embodiments, ascending doses of the anti-pruritus agent are administered during the titration until a steady state is achieved in the patient. In some embodiments, ascending doses of the anti-pruritus agent are administered during the titration until an effective amount of 15 mg, 30 mg, 60 mg, 90 mg, 120 mg, 180 mg, 240 mg or 360 mg is achieved in the patient.

In one embodiment, the titration is initiated with a dose of about 15 mg once or twice a day. In another embodiment, the titration is initiated with a dose of about 30 mg once or twice a day. In some embodiments, the titration comprises administering the anti-pruritus agent in increments ranging from about 15 mg to about 30 mg. In some embodiments, the titration comprises administering the anti-pruritus agent in increments ranging from about 15 mg to about 60 mg. In some embodiments, titration twice a day is with an AM dosage and a PM dosage, wherein the PM dosage is higher than or the same as the AM dosage.

In accordance with some embodiments of the present disclosure, the rate of adverse events after the treatment with the anti-pruritus agent is substantially the same as the rate of adverse events after administering a placebo for the same period of time. In some embodiments, the rate of liver-associated adverse events after the treatment with the anti-pruritus agent is substantially the same as the rate of adverse events after administering a placebo for the same period of time.

According to some embodiments of the present disclosure, clinical studies show that patients treated with an anti-pruritus agent experience a statistically significant reduction of itch compared to patients treated with a placebo. In some embodiments, the statistically significant reduction of itch is indicated by a p value of less than or equal to about 0.05. In some embodiments, the patient with moderate or severe baseline itch prior to the treatment experiences mild itch after the treatment.

According to some embodiments of the present disclosure, after the treatment the patient experiences a reduction of itch that is characterized by at least about a 30%, 40%, or 50% decline in worst itch intensity Numerical Rating Scale (NRS) value. In some embodiments, after the treatment the patient experiences a reduction of itch that is characterized by at least about a 30%, 40%, or 50% decline in average itch intensity Numerical Rating Scale (NRS) value. In some embodiments, after the treatment the patient experiences a reduction of itch that is characterized by at least 3 point decline or at least 4 point decline in the worst itch intensity NRS. In some embodiments, after the treatment the patient experiences a reduction of itch that is characterized by an at least 3 point decline or an at least 4 point decline in the average itch intensity NRS

According to some embodiments of the present disclosure, after the treatment the patient experiences a reduction of itch that is characterized by at least about a one category (or one unit) decline in intensity of the itchy Verbal Rating Scale (VRS) value.

According to some embodiments of the present disclosure, after the treatment the patient experiences a reduction of itch that is characterized by at least about a 10%, 20%, or 30% improvement in ItchyQoL′ total scale score or in any of the respective sub scales of Symptom Subscale score, Functional Subscale score, or Emotion Subscale score.

According to some embodiments of the present disclosure, after the treatment the patient experiences a reduction of itch that is characterized by at least about a 10%, 20%, or 30% improvement in Patient Benefit Index—pruritus version (PBI-P) scale.

In accordance with some embodiments of the present disclosure, the method of treating pruritus does not produce a substantial aquaretic effect.

In some embodiments, the method of treating pruritus further includes administering at least one additional antipruritic drug. In some embodiments, at least one additional antipruritic drug is selected from the group consisting of antihistamines (for example, loratadine), corticosteroids (for example, prednisone), capsaicin, calcineurin inhibitors (for example, tacrolimus), antibiotics (for example, tetracycline), anti-convulsants (for example, gabapentin), immunosuppressants (for example, methotrexate), anti-depressants (for example, amitriptyline), neuroleptics (for example, clozapine), benzodiazepine (for example, diazepam), serotonin antagonists, or immunomodulators (for example, thalidomide). In some embodiments, the method of treating pruritus further includes administering cholestyramine. In some embodiments, the method of treating pruritus further includes administering rifampicin.

In some embodiments, the anti-pruritus agent is in the form of an extended release oral dosage form.

In some embodiments, the anti-pruritus agent is administered in a formulation comprising nalbuphine hydrochloride, mannitol, hydroxypropyl cellulose, locust bean gum, xanthan gum, calcium sulfate dihydrate, and magnesium stearate.

The present methods, and advantages thereof, are further illustrated by the following non-limiting detailed description, including the Examples.

### Definitions

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “adverse event” (AE) as used herein is defined as any untoward medical occurrence in a clinical investigation patient reported on or after the first screening date. An AE does not necessarily have to have a causal relationship with the treatment. An AE can therefore be any unfavorable and unintended sign (including an abnormal laboratory finding), symptom whether or not related to the medicinal (investigational) product, or disease temporally associated with the use of a medicinal (investigational) product. Typical adverse events include nausea, vomiting, somnolence, and dizziness. In accordance with the present disclosure, the rate of adverse events after the treatment is substantially the same as the rate of adverse events after administering a placebo for the same period of time.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “chronic pruritus” is used in this disclosure to mean pruritus that lasts for at least 6 weeks.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt, solvate or ester thereof, that, when administered to a patient, is capable of performing the intended result. For example, an effective amount of an anti-pruritic agent is that amount which is required to reduce at least one symptom of pruritus in a patient, e.g. the amount required to reduce the itching sensation in a patient. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “treating” as used herein with regard to a patient, refers to improving at least one symptom of the patient's disorder. Treating can be curing, improving, or at least partially ameliorating a disorder.

The term “therapeutic effect” as used herein refers to a desired or beneficial effect provided by the method and/or the composition. For example, the method for treating pruritus provides a therapeutic effect when the method reduces at least one symptom of pruritus, e.g., itching sensation, in a patient.

### DETAILED DESCRIPTION

According to the present disclosure, pruritus includes any itchy or pruritic condition, e.g., a sensation that causes the desire to scratch.

Pruritus is a common and debilitating symptom of chronic liver disease. Chronic liver disease may be characterized as cholestatic or non-cholestatic. Cholestatic liver disease (or cholestasis) is an impairment of bile formation or bile flow. Non-cholestatic liver disease is liver disease that does not result in cholestasis (i.e., hepatocellular injury that is not severe enough to disrupt the normal hepatocellular excretion of bile into the biliary ductile system and therefore is not associated with elevated levels of bilirubin circulating in the blood). According to one study, about 40% of all chronic liver disease patients (cholestatic and non-cholestatic) report pruritus and about 60% of patients reporting pruritus do not respond to treatment with oral and/or topical antipruritic agents (S. Oeda, et al., Prevalence of pruritus in patients with chronic liver disease: A multicenter study, Hepatology Research, 2018, 48: E252-E262).

Despite its common occurrence, the etiology of pruritus associated with liver disease is poorly understood. Some sources, including Lindor, et al., Primary Biliary Cirrhosis, Hepatology, July 2009, 291-308, suggest that itch associated with cholestatic liver disease may be related to an increased opioidergic tone that results from decreased hepatic clearance of endogenous opioids. However, other sources, such as Kremer, et al., Pathogenesis and Treatment of Pruritus in Cholestasis Drugs, 2008; 68 (15), 2163-2182, acknowledge that there is no correlation between endogenous opioid levels and itch intensity in cholestatic liver disease patients.

European Association for the Study of Liver Itch (EASL) guidelines recommend cholestyramine as a first-line treatment, rifampicin (a pregnane X receptor agonist) as a second-line treatment and an oral opiate antagonist as a third-line treatment to relieve pruritus associated with cholestatic liver disease and primary biliary cholangitis (a.k.a., primary biliary cirrhosis (PBC)), a type of cholestatic liver disease. (EASL Clinical Practice Guidelines: Management of cholestatic liver diseases, J. Hepatology, 2009, 51, 237-267; EASL Clinical Practice Guidelines: The diagnosis and management of patients with primary biliary cholangitis, J. Hepatology, 2017, 67, 145-172). The use of oral opiate antagonists provides “disappointing” therapeutic results and is associated with opiate withdrawal-like symptoms as well as reduced pain threshold and confusion (2009 EASL guidelines at 258). Nalfurafine, a κ-opioid receptor agonist, is approved to treat pruritus associated with cholestatic liver disease in Japan. However, there is no FDA-approved therapy for treating pruritus associated with liver disease.

In one aspect, the present disclosure provides a method of treating pruritus comprising administering an effective amount of an anti-pruritus agent for at least about a week to a patient in need of such treatment, wherein the anti-pruritus agent is nalbuphine or a pharmaceutically acceptable salt, solvate or ester thereof. In accordance with some embodiments of the present disclosure, at least about 15 mg, 30 mg, 60 mg, 90 mg, 120 mg, or 180 mg of the anti-pruritus agent is administered.

In another embodiment, methods of the present disclosure are used for the treatment of pruritus associated with liver disease. In some embodiments, Nalbuphine HCl is used or indicated for the treatment of itch in patients with chronic pruritus associated with liver disease. In some embodiments, Nalbuphine HCl is used or indicated for the treatment of itch in patients with pruritus associated with liver disease wherein the patients do not have a bile duct obstruction.

In another embodiment, methods of the present disclosure are used for the treatment of pruritus associated with obstructive cholestasis secondary to bile duct obstruction due to non-hepatic tissue disease. In some embodiments, Nalbuphine HCl is used or indicated for the treatment of itch in patients with chronic pruritus associated with obstructive cholestasis secondary to bile duct obstruction due to non-hepatic tissue disease. In some embodiments, Nalbuphine HCl is used or indicated for the treatment of itch in patients with pruritus associated with obstructive cholestasis secondary to bile duct obstruction due to non-hepatic tissue disease wherein the obstruction is caused by a condition selected from the group consisting of pancreatic cancer, pancreatitis, congenital or acquired biliary strictures, lymph node obstruction such as from lymphomas or bile duct stones.

In another embodiment, methods of the present disclosure are used for the treatment of pruritus associated with cholestatic liver disease. In some embodiments, the cholestatic liver disease is primary sclerosing cholangitis. In some embodiments, the cholestatic liver disease is primary biliary cholangitis.

In another embodiment, methods of the present disclosure are used for the treatment of pruritus associated with non-cholestatic liver disease.

In some embodiments, the methods of the present disclosure are used to treat pruritus associated with a liver disease selected from infectious hepatitis; cirrhotic liver disease, drug-induced liver disease, idiopathic portal hypertension, congenital malformations or genetic diseases affecting liver function, sarcoidosis, primary or metastatic neoplasm involvement of the liver and autoimmune hepatitis-cholangitis (Overlap syndrome).

In some embodiments, the methods of the present disclosure are used to treat pruritus associated with infectious hepatitis. In some embodiments, the infectious hepatitis is selected from hepatitis C (HCV) and hepatitis B (HBV). In some embodiments, the HCV is selected from chronic HCV and HCV post-sustained virologic response. In some embodiments, the hepatitis B is selected from inactive HBV in a carrier and active HBV infection.

In some embodiments, the methods of the present disclosure are used to treat pruritus associated with cirrhotic liver disease. In some embodiments, the cirrhotic liver disease is selected from alcoholic liver disease, autoimmune hepatitis, and non-alcoholic fatty liver disease.

In some embodiments, the methods of the present disclosure are used to treat pruritus associated with a liver disease selected from drug-induced liver disease, idiopathic portal hypertension, congenital malformations or genetic diseases affecting liver function, sarcoidosis, primary or metastatic neoplasm involvement of the liver and autoimmune hepatitis-cholangitis (Overlap syndrome).

In some embodiments, the methods of the present disclosure are used to treat pruritus associated with a liver disease wherein patient's serum levels of endogenous opioids are elevated compared to normal serum levels. In some embodiments, the endogenous opioid is one or more endogenous μ-opioid receptor agonists. In some embodiments, the endogenous μ-opioid receptor agonist is selected from enkephalin and β-endorphin.

According to the present disclosure, the anti-pruritic agent is administered on a once or twice a day basis to provide effective relief of the symptoms of pruritus associated with liver disease (or obstructive cholestasis secondary to bile duct obstruction due to non-hepatic tissue disease) that is not effectively relieved by other therapies (i.e., the pruritus is refractory to other treatments).

In some embodiments, the methods of the present disclosure are used to treat pruritus associated with a liver disease (or obstructive cholestasis secondary to bile duct obstruction due to non-hepatic tissue disease) where the pruritus is refractory to treatment with other anti-pruritus agents. In some embodiments, the pruritus is refractory to treatment with an anti-pruritus agents selected from antidepressants, serotonin antagonists, serotonin reuptake inhibitors or antihistamines. In some embodiments, the pruritus is refractory to treatment with sertraline.

In some embodiments, the methods of the present disclosure are used to treat pruritus associated with a liver disease (or obstructive cholestasis secondary to bile duct obstruction due to non-hepatic tissue disease) where the pruritus is refractory to treatment with bile sequestrants. In some embodiments, the pruritus is refractory to treatment with a bile sequestrant selected from the group consisting of cholestyramine, colestipol and colesevelam. In some embodiments, the pruritus is refractory to treatment with cholestyramine.

In some embodiments, the methods of the present disclosure are used to treat pruritus associated with a liver disease (or obstructive cholestasis secondary to bile duct obstruction due to non-hepatic tissue disease) where the pruritus is refractory to treatment with one or more pregnane X receptor agonists. In some embodiments, the pruritus is refractory to treatment with rifampicin.

In some embodiments, the methods of the present disclosure are used to treat pruritus associated with a liver disease (or obstructive cholestasis secondary to bile duct obstruction due to non-hepatic tissue disease) where the pruritus is refractory to treatment with μ-opioid antagonists. In some embodiments, the pruritus is refractory to treatment with a μ-opioid antagonist selected from the group consisting naltrexone and naloxone.

In some embodiments, the methods of the present disclosure are used to treat pruritus associated with a liver disease (or obstructive cholestasis secondary to bile duct obstruction due to non-hepatic tissue disease) where the pruritus is refractory to treatment with κ-opioid agonists. In some embodiments, the pruritus is refractory to treatment with nalfurafine.

In accordance with some embodiments of the present disclosure, the method provides a therapeutic effect without producing a substantial adverse event. In some embodiments, the rate of adverse events after the treatment with the anti-pruritus agent is substantially the same as the rate of adverse events after administering a placebo for the same period of time. In some embodiments, the rate of liver-associated adverse events (such as elevated serum levels of liver function enzymes (i.e., serum alkaline phosphatase (“AP”), gamma-glutamyltranspeptidase (“GGT”), serum aminotransferases (alanine transaminase (“ALT”) and/or aspartate transaminase (“AST”)) after the treatment with the anti-pruritus agent is substantially the same as the rate of adverse events after administering a placebo for the same period of time.

In accordance with some embodiments of the present disclosure, the method of treating pruritus does not produce a substantial aquaretic effect.

In some embodiments of the present disclosure, the patient treated for pruritus is a pediatric patient. In some embodiments of the present disclosure, the patient treated for pruritus is a geriatric patient.

Nalbuphine

Nalbuphine as employed in the present methods can form a part of a pharmaceutical composition by combining nalbuphine, or a pharmaceutically acceptable salt, solvate or ester thereof, with a pharmaceutically acceptable carrier. Additionally, the compositions can include an additive selected from the group consisting of adjuvants, excipients, diluents, release-modifying agents and stabilizers. The composition can be an immediate release formulation, a delayed release formulation, a sustained release formulation or an extended release formulation.

Nalbuphine HCl (17-(cyclobutylmethyl)-4,5α-epoxymorphinan-3, 6α, 14-triol, hydrochloride) is a synthetic opioid. Structurally, nalbuphine is a derivative of 14 hydroxymorphine.

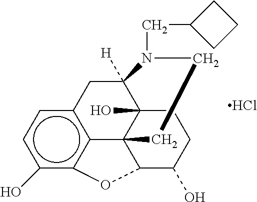

Nalbuphine HCl is currently available only as a generic medication in an injectable form. An injectable form of nalbuphine has been available as an approved drug formulation since 1978. Nubain® was the innovator brand injectable form of nalbuphine on which the presently sold generic bioequivalent injectable formulations are based. The injectable formulation is currently approved for use in the relief of moderate to severe pain, a supplement to balanced anesthesia, for pre-operative and post-operative analgesia and obstetrical analgesia during labor and delivery.

The present disclosure also includes pharmaceutically acceptable esters of the anti-pruritus agent. The term “ester” denotes a derivative of the agent containing an ester functional group (as described herein), which is capable of releasing the agent when the ester form is administered to a patient. Release of the active ingredient occurs in vivo. Pharmaceutically acceptable esters can be prepared by techniques known to one skilled in the art. These techniques generally modify appropriate functional groups in a given compound. These modified functional groups however regenerate original functional groups by metabolism of the compound in vivo. Esters include compounds wherein a hydroxy, carboxylic, or a similar group is modified.

Suitable pharmaceutically acceptable esters for a hydroxyl group include inorganic esters such as phosphate esters and α-acyloxyalkyl ethers and related compounds which, as a result of in vivo hydrolysis of the ester, provide the parent hydroxy group. In vivo hydrolyzable ester forming groups for hydroxy include alkanoyl (e.g., C₁₋₁₀ linear, branched or cyclic alkyl), benzoyl, phenylacetyl and substituted benzoyl and phenylacetyl, alkoxycarbonyl (to give alkyl carbonate esters), dialkylcarbamoyl and N—(N, N-dialkylaminoethyl)-N-alkylcarbamoyl (to give carbamates), N, N-dialkylaminoacetyl and carboxyacetyl.

In some embodiments, the nalbuphine used in the formulations and methods of the present disclosure is a pharmaceutically acceptable co-crystal of nalbuphine.

Formulations

The methods of the present disclosure can employ various formulations for administration to patients, e.g., humans and animals in unit dosage forms, such as tablets, capsules, pills, powders, granules, sterile parenteral solutions or suspensions, and oral solutions or suspensions, and oil-water emulsions containing suitable quantities of an anti-pruritic agent, e.g., nalbuphine, or pharmaceutically acceptable salts or esters thereof.

Oral pharmaceutical dosage forms can be either solid or liquid. The solid dosage forms can be tablets, capsules, granules, and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In other embodiments, the oral dosage form may be an osmotic-controlled release oral delivery system (OROS). In other embodiments, the oral dosage form may include matrix-embedded dosage forms or related devices. In some embodiments, the present oral dosage forms may include orally-disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules.

Aqueous solutions include, for example, elixirs and syrups. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Emulsions can be either oil-in water or water-in-oil. Pharmaceutically acceptable carriers used in emulsions are nonaqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, can include organic acids and a source of carbon dioxide. Coloring and flavoring agents can be used in all of the above dosage forms.

Parenteral administration of the formulations of the present disclosure includes intravenous, subcutaneous and intramuscular administrations of immediate, sustained (e.g., depot), extended, and/or modified release formulations (e.g., as described herein). Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

The concentration of the pharmaceutically active compound can be adjusted so that an injection provides an effective amount to produce the desired pharmacological effect. The exact dose depends on the age, weight and condition of the patient or animal, as is known in the art. The unit-dose parenteral preparations are packaged in an ampoule or a syringe with a needle. All preparations for parenteral administration must be sterile, as is known and practiced in the art. Illustratively, intravenous or intra-arterial infusion of a sterile aqueous solution containing an anti-pruritic agent is an effective mode of administration.

Pharmaceutical dosage forms for rectal administration can be rectal suppositories, capsules and tablets for systemic effect. Rectal suppositories as used herein mean solid bodies for insertion into the rectum which melt or soften at body temperature releasing the pharmacologically and/or therapeutically active ingredients contained in the composition of this disclosure. Pharmaceutically acceptable substances utilized in rectal suppositories are bases or vehicles and agents to raise the melting point. Examples of bases include cocoa butter (theobroma oil), glycerin-gelatin, carbowax, polyoxyethylene glycol and mixtures of mono-, di- and triglycerides of fatty acids. Combinations of the various bases can be used. Agents to raise the melting point of suppositories include spermaceti and wax. Rectal suppositories can be prepared either by the compressed method or by molding. The typical weight of a rectal suppository is about 2 to 3 gm. Tablets and capsules for rectal administration can be manufactured using the same pharmaceutically acceptable substance and by the same methods as for formulations for oral administration.

The compositions can be suspended in micronized or other suitable form or can be derivatized to produce a more soluble active product. The form of the resulting composition depends upon a number of factors, including the intended mode of administration and the solubility of the anti-pruritic agent in the selected carrier or vehicle. The effective concentration is sufficient for treating or alleviating pruritus, and can be empirically determined. The concentration is generally greater than the concentration for systemic administration of the compound.

The resulting mixture can be a solution, suspension, emulsion or the like, and can be formulated as a cream, gel, ointment, emulsion, solution, elixir, lotion, suspension, tincture, paste, foam, aerosol, irrigation, spray, suppository, bandage, or any other formulation suitable for topical administration. Modes of administration can include topical application to the skin, scalp, eyes, and/or nasal, buccal or sublingual mucosa.

Pharmaceutical and cosmetic carriers or vehicles suitable for administration of the compositions include any such carriers known to those skilled in the art to be suitable for the particular mode of administration. The anti-pruritic agent can be included in the carriers in amounts sufficient to exert a therapeutically useful effect without serious toxic effects on the treated individual.

To formulate these compositions, a weight fraction of an anti-pruritic agent is dissolved, suspended, dispersed or otherwise mixed in a selected vehicle at an effective concentration such that the pruritic condition is relieved or ameliorated. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream (USP), and hydrophilic ointment (USP).

The compositions employed in the present methods can relieve pruritus when applied to the skin. Relief can be temporary or permanent, and can even be evident after a single dose of the composition. When the composition is administered in a form other than a topical preparation, it should be administered in an amount sufficient to provide relief from pruritus that is within safety guidelines established by the FDA. Determining the appropriate amount to administer to a patient is within the skill of the person of ordinary skill in the art in association with teachings provided by the present disclosure.

Solutions of the compositions of this disclosure intended for topical administration contain an amount of the composition effective to deliver an anti-pruritic amount, typically at a concentration of between about 0.01% w/w to about 5% w/w. The balance of the solution is water, a suitable organic solvent or other suitable solvent or buffer. These compositions that are formulated as solutions or suspensions can be applied to the skin, or can be formulated as an aerosol or foam and applied to the skin as a spray-on. The aerosol compositions typically contain from 25% to 80% w/w, preferably from 30% to 50% w/w, of a suitable propellant. Gel compositions can be formulated by simply admixing a suitable thickening agent to the solution or suspension.

Compositions of solid forms intended for topical application can be formulated as stick-type compositions intended for application to the lips or other parts of the body. Such compositions contain an effective amount of an anti-pruritic agent, e.g. nalbuphine or a pharmaceutically acceptable salt, solvate or ester thereof. The amount of the anti-pruritic agent present is typically from about 0.01% w/w to about 5% w/w. The solids also contain from about 40% to 98% w/w, preferably from about 50% to 90% w/w, of emollients. This composition can further contain from 1% to 20% w/w, preferably from 5% to 15% w/w, of a suitable thickening agent, and, if desired or needed, emulsifiers and water or buffers.

Sustained Release

Nalbuphine formulations that can be employed in the present methods include oral sustained release nalbuphine formulations as described in U.S. Patent Publication Nos. 2019/0117576, 2019/0099416, 2015/0359789 2009/0030026, and 2007/0048376; and PCT Publication Nos. 2015/192071 and 2007/025005; each of which is incorporated herein by reference in their entireties.

“Sustained release” or “extended release” means that the nalbuphine or pharmaceutically acceptable salt, solvate or ester thereof is released from the formulation at a controlled rate so that therapeutically beneficial blood levels (but below toxic levels) of the nalbuphine or pharmaceutically acceptable salt, solvate or ester thereof are maintained over an extended period of time. Alternatively, “sustained release” or “extended release” means that the desired pharmacologic effect is maintained over an extended period of time.

The half-life of nalbuphine injectable formulations (i.e., IV or IM or SC) has been reported to be relatively short, only about 2-3 hours. In some embodiments, the present methods can employ oral sustained release formulations of nalbuphine including an anti-pruritic effective amount of nalbuphine or a pharmaceutically acceptable salt, solvate or ester thereof. The oral sustained release formulations can provide a controlled release and a lower Cₘₐₓ of the anti-pruritus agent over a longer period than observed for bolus injections or immediate release oral formulations (e.g., at least about 8-12 hours). Reducing the frequency of dosing provides the potential for enhanced patient convenience and compliance with the present methods. The lower dosing frequency also has the potential to provide reduced side effects because the patient may be exposed to lower peak concentrations of agent over time.

Without wishing to be bound by a particular theory, the longer than expected duration of anti-pruritic effect is attributed to the enterohepatic recirculation of nalbuphine. Nalbuphine forms a glucuronic acid or other type of conjugated metabolite in vivo through enzymatic reaction with an enzyme system such as UDP-glucuronyl transferase. It is also possible that enterohepatic recirculation also occurs when parent drug in the bile is released from the gallbladder into the intestine and reabsorbed. Once formed, the conjugated nalbuphine product is thought to be transported into the gastrointestinal tract via biliary secretion whereby the drug conjugate is cleaved liberating nalbuphine, which can be reabsorbed from the intestine. The sustained release formulation can improve the duration of anti-pruritic effect, by more slowly releasing nalbuphine into the in vivo system and allowing more drug to be conjugated and therefore available for recirculation and later reabsorption from the intestine.

The present methods can employ compositions including nalbuphine or a pharmaceutically acceptable salt, solvate or ester thereof and a sustained release delivery system. The sustained release delivery system includes (i) at least one hydrophilic compound, at least one cross-linking agent, and at least one pharmaceutical diluent; (ii) at least one hydrophilic compound, at least one cross-linking agent, at least one pharmaceutical diluent, and at least one cationic cross-linking agent different from the first cross-linking agent; or (iii) at least one hydrophilic compound, at least one cationic cross-linking compound, and at least one pharmaceutical diluent. Alternatively, in other embodiments, the present methods can employ compositions including nalbuphine or a pharmaceutically acceptable salt, solvate or ester thereof and a sustained release delivery system, which may employ a hydrophobic compound in a sustained release system.

The nalbuphine can be homogeneously dispersed in the sustained release delivery system. In some embodiments, the nalbuphine or pharmaceutically acceptable salt, solvate or ester thereof is present in the composition in an amount of about 1 mg to about 240 mg; about 1 mg to about 150 mg; about 1 mg to about 125 mg; or about 1 mg to about 100 mg. In some embodiments, the nalbuphine or pharmaceutically acceptable salt, solvate or ester thereof is present in the composition in an amount of about 5 mg to about 80 mg; about 10 mg to about 70 mg; about 15 mg to about 60 mg; about 40 mg to about 80 mg; about 50 mg to about 70 mg; or about 45 mg to about 60 mg. In one embodiment, the nalbuphine or pharmaceutically acceptable salt, solvate or ester thereof is present in the composition in an amount of about 15 mg, about 20 mg, about 25 mg, about 30 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, or about 240 mg. In another embodiment, the nalbuphine or pharmaceutically acceptable salt thereof is present in the composition in an amount of about 15 mg, about 30 mg, about 45 mg, about 60 mg, about 90 mg, about 120 mg, or about 180 mg.

In yet another embodiment, the pharmaceutically acceptable salt of nalbuphine, e.g., nalbuphine HCl, is present in the composition in an amount of about 15 mg, about 30 mg, about 60 mg, about 90 mg, about 120 mg, or about 180 mg. For compositions comprising a pharmaceutically acceptable salt of nalbuphine, the amount of nalbuphine in said compositions may be expressed as the Equivalent Amount of Nalbuphine Free Base, which is the calculated amount of nalbuphine free base in the composition based on the actual amount of the pharmaceutically acceptable salt of nalbuphine in the composition. The amount of the Equivalent Amount of Nalbuphine Free Base in a composition will vary within the manufacturing process, and the compositions of the present disclosure encompass pharmaceutically-acceptable deviations (i.e., FDA-acceptable) from the nalbuphine content that is recited in the present disclosure.

The following table shows the Equivalent Amount of Nalbuphine Free Base for compositions containing 15 mg, 30 mg, 60 mg, 90 mg, 120 mg, 180 mg and 240 mg of nalbuphine HCl:

| | |
|---|---|
| Amount of | Equivalent Amount of |
| nalbuphine HCl | Nalbuphine Free Base |

| | | |
|---|---|---|
| 15 | mg | 13.6¹ |
| 30 | mg | 27.2 |
| 60 | mg | 54.4 |
| 90 | mg | 81.6 |
| 120 | mg | 108.8 |
| 180 | mg | 163.2 |
| 240 | mg | 217.6 |

¹The amount of Equivalent Amount of Nalbuphine Free Base is rounded to the nearest 0.1 decimal place using the equation below.

Throughout the present disclosure, the amount of nalbuphine in a composition is generally expressed in terms of the amount of nalbuphine hydrochloride present in a composition. However, the present disclosure contemplates embodiments where the nalbuphine is present in another nalbuphine form (such as a different pharmaceutically acceptable salt and/or ester) and provides about the same Equivalent Amount of Nalbuphine Free Base as the embodiments that are expressly described herein. For example, about 251 mg of nalbuphine citrate (FW=549.57 g/mol) provides about the same Equivalent Amount of Nalbuphine Free Base as about 180 mg of nalbuphine hydrochloride. The Equivalent Amount of Nalbuphine Free Base in said compositions may be calculated by the following formula:

$$\text{Equivalent Amount of Nalbuphine Free Base} = \frac{\begin{matrix}\text{Mass of Pharmaceutically Acceptable Salt (g)} \times \\ 357.45 ( \text{Formula Weight of Nalbuphine Free Base} , \frac{g}{mol} )\end{matrix}}{\text{Formula Weight of Pharmaceutically Acceptable Salt} ( \frac{g}{mol} )}$$

The Equivalent Amount of Nalbuphine Free Base content of the dosage form calculated using the equation above may be adjusted by a pharmaceutically acceptable amount (for example, within an amount permitted by FDA safety standards, which in some embodiments is 1% or less of the calculated Equivalent Amount of Nalbuphine Free Base) to allow product labeling using a whole number integer when referencing the dosage strength. For example, the calculated Equivalent Amount of Nalbuphine Free Base for 240 mg of nalbuphine hydrochloride is 217.6 mg. According to the present disclosure, the nalbuphine content of the composition may be adjusted for a product labelling of 216 mg of Equivalent Amount of Nalbuphine Free Base.

In some embodiments, the sustained release delivery system is present in the composition in an amount from about 10 mg to about 420 mg; from about 25 mg to about 225 mg; from about 21 mg to about 198 mg; from about 80 mg to about 200 mg; from about 80 mg to about 220 mg; from about 90 mg to about 210 mg; from about 100 mg to about 200 mg; from about 110 mg to about 190 mg; from about 120 mg to about 180 mg; from about 130 mg to about 170 mg; from about 140 mg to about 160 mg; from about 30 mg to about 60 mg; from about 60 mg to about 180 mg; from about 30 mg to about 180 mg; from about 75 mg to about 150 mg; from about 80 mg to about 160 mg; from about 90 mg to about 150 mg; from about 100 mg to about 140 mg; from about 110 mg to about 130 mg, from about 100 mg to about 300 mg; from about 200 mg to about 300 mg or from about 200 mg to about 250 mg. In one embodiment, the sustained release delivery system is present in the composition in an amount from about 75 mg to about 150 mg.

In some embodiments, the sustained release delivery system is present in the composition in an amount of about 15 mg, about 30 mg, about 60 mg, about 75 mg, about 80 mg, about 90 mg, about 100 mg, about 110 mg, about 112 mg, about 115 mg, about 117 mg, about 120 mg, about 125 mg, about 130 mg, about 135 mg, about 140 mg, about 145 mg, about 150 mg, about 160 mg, about 170 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 300 mg, about 320 mg, about 340 mg, about 360 mg, about 380 mg, about 400 mg or about 420 mg. In another embodiment, the sustained release delivery system is present in the composition in an amount of about 112 mg.

The ratio of nalbuphine or pharmaceutically acceptable salt, solvate or ester thereof in the compositions to the sustained release delivery system is generally from about 4:1 to about 1:25. In some embodiments, the ratio of nalbuphine or pharmaceutically acceptable salt, solvate or ester thereof to the sustained release delivery system is generally from about 2.5:1 to about 1:4. In some embodiments, the ratio of nalbuphine or pharmaceutically acceptable salt, solvate or ester thereof to the sustained release delivery system is generally from about 5:1 to about 1:5, about 4:1 to about 1:4, about 3:1 to about 1:3, about 2:1 to about 1:2, about 1:1 to about 1:5, about 1:1 to about 1:4, about 1:1 to about 1:3, about 1:1 to about 1.2, and about 1:2 to about 1:3. In some embodiments, the ratio of nalbuphine or pharmaceutically acceptable salt, solvate or ester thereof to the sustained release delivery system is about 1:1, about 1:2, about 1:2.5, about 1:3, about 1:4, or about 1:5.

In one embodiment, at least one hydrophilic compound is present in the sustained release delivery system in an amount of about 5% to about 80% by weight; the at least one cross-linking agent is present in the sustained release delivery system in an amount of about 0.5% to about 80% by weight; and the at least one pharmaceutical diluent is present in the sustained release delivery system in an amount of about 20% to about 80% by weight. In another embodiment, the at least one hydrophilic compound is present in the sustained release delivery system in an amount of about 8% to about 31% by weight; the at least one cross-linking agent is present in the sustained release delivery system in an amount of about 12% to about 47% by weight; and the at least one pharmaceutical diluent is present in the sustained release delivery system in an amount of about 20% to about 78% by weight. In another embodiment, the at least one hydrophilic compound is present in the sustained release delivery system in an amount of about 10% to about 20% by weight; the at least one cross-linking agent is present in the sustained release delivery system in an amount of about 15% to about 25% by weight; and the at least one pharmaceutical diluent is present in the sustained release delivery system in an amount of about 50% to about 85% by weight. In some embodiments, the at least one hydrophilic compound is present in the sustained release delivery system in an amount of about 8%, about 9%, about 10%, about 11%, about 12%, about 13%, about 14%, about 15%, about 16%, about 17%, about 18%, about 19%, about 20%, about 22%, about 24%, about 26%, about 28%, about 30%, about 32%, about 34%, or about 36% by weight; the at least one cross-linking agent is present in the sustained release delivery system in an amount of about 10%, about 11%, about 12%, about 13%, about 14%, about 15%, about 16%, about 17%, about 18%, about 19%, about 20%, about 22%, about 24%, about 26%, about 28%, about 30%, about 32%, about 33%, about 34%, or about 35% by weight; and the at least one pharmaceutical diluent is present in the sustained release delivery system in an amount of about 40%, about 45%, about 50%, about 55%, about 60%, about 65%, about 70%, about 80%, or about 85% by weight.

In some embodiments, the at least one hydrophilic compound is present in the sustained release delivery system in an amount of about 10%, about 11%, about 12%, about 13%, about 14%, about 15%, about 16%, about 17%, about 18%, about 19%, or about 20% by weight; the at least one cross-linking agent is present in the sustained release delivery system in an amount of about 15%, about 16%, about 17%, about 18%, about 19%, about 20%, or about 22% by weight; and the at least one pharmaceutical diluent is present in the sustained release delivery system in an amount of about 55%, about 60%, about 65%, about 70%, about 80%, or about 85% by weight. In one embodiment, the at least one hydrophilic compound is present in the sustained release delivery system in an amount of about 8%, about 12%, or about 20% by weight; the at least one cross-linking agent is present in the sustained release delivery system in an amount of about 12%, about 18%, or about 30% by weight; and the at least one pharmaceutical diluent is present in the sustained release delivery system in an amount of about 40%, about 60%, or about 70% by weight.

In one embodiment, nalbuphine is in the form of any pharmaceutically acceptable salt known in the art. Exemplary pharmaceutically acceptable salts include without limitation hydrochloric, sulfuric, nitric, phosphoric, hydrobromic, maleic, malic, ascorbic, citric, tartaric, pamoic, lauric, stearic, palmitic, oleic, myristic, lauryl sulfuric, naphthalenesulfonic, linoleic, linolenic acid, and the like. One embodiment includes the hydrochloride salt of nalbuphine.

The sustained release delivery system includes at least one hydrophilic compound. The hydrophilic compound preferably forms a gel matrix that releases the nalbuphine or the pharmaceutically acceptable salt, solvate or ester thereof at a sustained rate upon exposure to liquids. The rate of release of the nalbuphine or the pharmaceutically acceptable salt, solvate or ester thereof from the gel matrix depends on the drug's partition coefficient between the components of the gel matrix and the aqueous phase within the gastrointestinal tract. The weight ratio of nalbuphine to hydrophilic compound is generally in the range of about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, and about 2:1 to about 1:2. In some embodiments, the weight ratio of nalbuphine to hydrophilic compound is in the range of about 10:1 to about 1:1, about 10:1 to about 2:1, about 9:1 to about 1:1, about 8:1 to about 1:1, about 7:1 to about 1:1, about 6:1 to about 1:1, about 5:1 to about 1:1, about 4:1 to about 1:1, about 3:1 to about 1:1, and about 2:1 to about 1:1. In some embodiments, the weight ratio of nalbuphine to hydrophilic compound is in the range of about 6:1 to about 1:1, about 5:1 to about 2:1, about 4:1 to about 3:1, about 4:1 to about 2:1, and about 5:1 to about 2:1. In some embodiments, the weight ratio of nalbuphine to hydrophilic compound is about 1:5, about 1:4.5, about 1:4.4, about 1:4, about 1:3.5, about 1:3.3, about 1:3, about 1:2.5, about 1:2, about 1:1, and about 1:1.5.

The sustained release delivery system generally includes the hydrophilic compound in an amount of about 5% to about 80% by weight. In some embodiments, the sustained release delivery system generally includes the hydrophilic compound in an amount of about 5% to about 30%, about 8% to about 31%, about 10% to about 20%, about 20% to about 60%, or about 40% to about 60% by weight. In one embodiment, the sustained release delivery system includes the hydrophilic compound in an amount of about 8% to about 31% by weight. In one embodiment, the sustained release delivery system includes the hydrophilic compound in an amount of about 10% to about 20% by weight. In some embodiments, the sustained release delivery system includes the hydrophilic compound in an amount of about 10%, about 11%, about 12%, about 13%, about 14%, about 15%, about 16%, about 17%, about 18%, about 19%, or about 20% by weight. In one embodiment, the sustained release delivery system includes the hydrophilic compound in an amount of about 12% by weight. In one embodiment, the sustained release delivery system includes the hydrophilic compound in an amount of about 8% by weight. In one embodiment, the sustained release delivery system includes the hydrophilic compound in an amount of about 20% by weight. In one embodiment, the sustained release delivery system includes the hydrophilic compound in an amount of about 28% by weight.

The hydrophilic compound is any pharmaceutically acceptable compound known in the art to be hydrophilic. Exemplary hydrophilic compounds include without limitation pharmaceutically acceptable gums, cellulose ethers, polyvinyl pyrrolidone, protein-derived compounds, and mixtures thereof. Exemplary gums include without limitation heteropolysaccharide gums and homopolysaccharide gums, such as xanthan, tragacanth, pectins, acacia, karaya, alginates, agar, guar, hydroxypropyl guar, carrageenan, locust bean gums, and gellan gums. Exemplary cellulose ethers include without limitation hydroxyalkyl celluloses and carboxyalkyl celluloses. In some embodiments, cellulose ethers include hydroxyethyl celluloses, hydroxypropyl celluloses, hydroxypropylmethyl-celluloses, carboxy methylcelluloses, and mixtures thereof. In some embodiments, the hydrophilic compound is a gum. In other embodiments, the hydrophilic compound is a heteropolysaccharide gum. In further embodiments, the hydrophilic compound is a xanthan gum or derivative thereof. Derivatives of xanthan gum include without limitation, for example, deacylated xanthan gum, the carboxymethyl esters of xanthan gum, and the propylene glycol esters of xanthan gum.

In another aspect, the sustained release delivery system further includes at least one cross-linking agent. In one embodiment, the cross-linking agent is a compound that is capable of cross-linking the hydrophilic compound to form a gel matrix in the presence of liquids. As used herein, “liquids” includes, for example, gastrointestinal fluids and aqueous solutions, such as those used for in vitro dissolution testing. The sustained release delivery system generally includes the cross-linking agent in an amount of about 0.5% to about 80% by weight. In one embodiment, the sustained release delivery system generally includes the cross-linking agent in an amount of about 12% to about 47% by weight. In another embodiment, the sustained release delivery system generally includes the cross-linking agent in an amount of about 20% to about 30% by weight. In one embodiment, the sustained release delivery system generally includes the cross-linking agent in an amount of about 15% to about 25% by weight. In some embodiments, the at least one cross-linking agent is present in the sustained release delivery system in an amount of about 15%, about 16%, about 17%, about 18%, about 19%, about 20%, about 21%, about 22%, about 23%, about 24%, or about 25% by weight. In one embodiment, the sustained release delivery system includes the cross-linking agent in an amount of about 18% by weight. In one embodiment, the sustained release delivery system includes the cross-linking agent in an amount of about 12% by weight. In one embodiment, the sustained release delivery system includes the cross-linking agent in an amount of about 30% by weight. In one embodiment, the sustained release delivery system includes the cross-linking agent in an amount of about 42% by weight.

Exemplary cross-linking agents include homopolysaccharides. Exemplary homopolysaccharides include without limitation galactomannan gums, such as guar gum, hydroxypropyl guar gum, and locust bean gum. In some embodiments, the cross-linking agent is a locust bean gum or a guar gum. In other embodiments, the cross-linking agent is an alginic acid derivative or hydrocolloid.

In some embodiments, when the sustained release delivery system includes at least one hydrophilic compound and at least one cross-linking agent, the weight ratio of hydrophilic compound to cross-linking agent is from about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, or about 1:2 to about 2:1. In some embodiments, the weight ratio of hydrophilic compound to cross-linking agent is about 1:5, about 1:4.5, about 1:4, about 1:3.5, about 1:3, about 1:2.5, about 1:2, about 1:1.5, and about 1:1.

When the sustained release delivery system includes at least one hydrophilic compound and at least one cross-linking agent, the weight ratio of the nalbuphine or pharmaceutically acceptable salt, solvate or ester thereof to the sum of the at least one hydrophilic compound and the at least one cross-linking agent is from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, or from about 2:1 to about 1:2. In some embodiments, the weight ratio of the nalbuphine or pharmaceutically acceptable salt, solvate or ester thereof to the sum of the at least one hydrophilic compound and the at least one cross-linking agent is from about 4:1 to about 1:1, from about 4:1 to about 1:1.5, from about 3:1 to about 1:1, or from about 2:1 to about 1:1. In one embodiment, the ratio of the nalbuphine or pharmaceutically acceptable salt, solvate or ester thereof to the sum of the at least one hydrophilic compound and the at least one cross-linking agent is about 5:1, about 4:1 (i.e., 1:0.25), about 3.5:1, about 3:1, about 2.5:1, about 2:1 (i.e., 1:0.5), about 1.9:1, about 1.8:1, about 1.7:1, about 1.6:1, about 1.5:1, about 1.4:1, about 1.3:1, about 1.2:1, about 1.1:1, about 1:1, about 1:1.5, about 1:2, about 1:3, about 1:4, and about 1:5.

The sustained release delivery system further includes one or more pharmaceutical diluents known in the art. Exemplary pharmaceutical diluents include without limitation monosaccharides, disaccharides, polyhydric alcohols and mixtures thereof. In some embodiments, pharmaceutical diluents include, for example, starch, mannitol, lactose, dextrose, sucrose, microcrystalline cellulose, sorbitol, xylitol, fructose, and mixtures thereof. In some embodiments, the pharmaceutical diluent is water-soluble. Nonlimiting examples of water-soluble pharmaceutical diluents include lactose, dextrose, sucrose, or mixtures thereof. The weight ratio of pharmaceutical diluent to hydrophilic compound is generally from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, or from about 1:2 to about 2:1. In some embodiments, the weight ratio of pharmaceutical diluent to hydrophilic compound is generally from about 9:1 to about 1:1.5. In some embodiments, the weight ratio of pharmaceutical diluent to hydrophilic compound is about 9:1, about 8.75:1, about 8.5:1, about 8.25:1, about 8:1, about 7.5:1, about 7:1, about 6.5:1, about 6:1, about 5.5:1, about 5:1, about 4.5:1, about 4:1, about 3.5:1, about 3:1, about 2.5:1, about 2:1, about 1.5:1, or about 1:1.

The sustained release delivery system generally includes one or more pharmaceutical diluents in an amount of about 20% to about 80%, about 30% to about 70%, about 40% to about 70%, or about 40% to about 60%. In one embodiment, the sustained release delivery system includes one or more pharmaceutical diluents in an amount of about 20% to about 70% by weight. In one embodiment, the sustained release delivery system includes one or more pharmaceutical diluents in an amount of about 50% to about 85% by weight. In some embodiments, the sustained release delivery system includes one or more pharmaceutical diluents in an amount of about 55%, about 60%, about 65%, about 70%, about 80%, or about 85% by weight. In one embodiment, the sustained release delivery system includes one or more pharmaceutical diluents in an amount of about 20% by weight. In one embodiment, the sustained release delivery system includes one or more pharmaceutical diluents in an amount of about 30% by weight. In one embodiment, the sustained release delivery system includes one or more pharmaceutical diluents in an amount of about 40% by weight. In one embodiment, the sustained release delivery system includes one or more pharmaceutical diluents in an amount of about 50% by weight. In one embodiment, the sustained release delivery system includes one or more pharmaceutical diluents in an amount of about 60% by weight. In one embodiment, the sustained release delivery system includes one or more pharmaceutical diluents in an amount of about 70% by weight.

In a further aspect, the sustained release delivery system includes one or more cationic cross-linking compounds. In some embodiments, the one or more cationic cross-linking compounds are used instead of the cross-linking agent. In some embodiments, the one or more cationic cross-linking compounds are used in addition to the cross-linking agent. In one embodiment, the one or more cationic cross-linking compounds are used in an amount sufficient to cross-link the hydrophilic compound to form a gel matrix in the presence of liquids. In some embodiments, the one or more cationic cross-linking compounds are present in the sustained release delivery system in an amount of about 0.5% to about 30%, about 0.5% to about 25%, about 0.5% to about 20%, about 0.5% to about 15%, about 0.5% to about 10%, or about 0.5% to about 5% by weight. In some embodiments, the one or more cationic cross-linking compounds are present in the sustained release delivery system in an amount of about 5% to about 20%, about 5% to about 15%, about 6% to about 14%, about 7% to about 13%, about 8% to about 12%, or about 9% to about 11% by weight. In some embodiments, the one or more cationic cross-linking compounds are present in the sustained release delivery system in an amount of about 5%, about 6%, about 7%, about 8%, about 9%, about 10%, about 11%, about 12%, about 13%, about 14%, or about 15% by weight. In one embodiment, the cationic cross-linking compound is present in the sustained release delivery system in an amount of about 10% by weight.

Exemplary cationic cross-linking compounds include without limitation monovalent metal cations, multivalent metal cations, and inorganic salts, including alkali metal and/or alkaline earth metal sulfates, chlorides, borates, bromides, citrates, acetates, lactates, and mixtures thereof. For example, the cationic cross-linking compound include without limitation one or more of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, or mixtures thereof.

When the sustained release delivery system includes at least one hydrophilic compound and at least one cationic cross-linking compound, the weight ratio of hydrophilic compound to cationic cross-linking compound ranges from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, or from about 1:2 to about 2:1. In one embodiment, the weight ratio of hydrophilic compound to cationic cross-linking compound ranges from about 1:3 to about 3:1. In some embodiments, the weight ratio of hydrophilic compound to cationic cross-linking compound is about 3:1, about 2.75:1, about 2.5:1, about 2.25:1, about 2:1, about 1.8:1, about 1.6:1, about 1.4:1, about 1.2:1, about 1:1, about 1:1.25, about 1:1.5, or about 1:2. In one embodiment, the weight ratio of hydrophilic compound to cationic cross-linking compound is about 1:1.25. In one embodiment, the weight ratio of hydrophilic compound to cationic cross-linking compound is about 1.2:1. In one embodiment, the weight ratio of hydrophilic compound to cationic cross-linking compound is about 2:1. In one embodiment, the weight ratio of hydrophilic compound to cationic cross-linking compound is about 2.8:1.

In one embodiment, the at least one hydrophilic compound is present in the sustained release delivery system in an amount of about 5% to about 80% by weight; the at least one cationic cross-linking agent is present in the sustained release delivery system in an amount of about 0.5% to about 30% by weight; and the at least one pharmaceutical diluent is present in the sustained release delivery system in an amount of about 20% to about 80% by weight. In another embodiment, the at least one hydrophilic compound is present in the sustained release delivery system in an amount of about 8% to about 30% by weight; the at least one cationic cross-linking agent is present in the sustained release delivery system in an amount of about 10% by weight; and the at least one pharmaceutical diluent is present in the sustained release delivery system in an amount of about 20% to about 70% by weight. In another embodiment, the at least one hydrophilic compound is present in the sustained release delivery system in an amount of about 5% to about 30% by weight; the at least one cationic cross-linking agent is present in the sustained release delivery system in an amount of about 5% to about 20% by weight; and the at least one pharmaceutical diluent is present in the sustained release delivery system in an amount of about 20% to about 85% by weight. In another embodiment, the at least one hydrophilic compound is present in the sustained release delivery system in an amount of about 10% to about 20% by weight; the at least one cationic cross-linking agent is present in the sustained release delivery system in an amount of about 5% to about 15% by weight; and the at least one pharmaceutical diluent is present in the sustained release delivery system in an amount of about 50% to about 85% by weight.

In some embodiments, the at least one hydrophilic compound is present in the sustained release delivery system in an amount of about 8%, about 9%, about 10%, about 11%, about 12%, about 13%, about 14%, about 15%, about 16%, about 17%, about 18%, about 19%, about 20%, about 22%, about 24%, about 26%, about 28%, or about 30% by weight; the at least one cationic cross-linking agent is present in the sustained release delivery system in an amount of about 5%, about 6%, about 7%, about 8%, about 9%, about 10%, about 11%, about 12%, about 13%, about 14%, about 15%, about 16%, about 17%, about 18%, about 19%, or about 20%, by weight; and the at least one pharmaceutical diluent is present in the sustained release delivery system in an amount of about 40%, about 45%, about 50%, about 55%, about 60%, about 65%, about 70%, about 80%, or about 85% by weight. In one embodiment, the at least one hydrophilic compound is present in the sustained release delivery system in an amount of about 10%, about 11%, about 12%, about 13%, about 14%, about 15%, about 16%, about 17%, about 18%, about 19%, or about 20% by weight; the at least one cationic cross-linking agent is present in the sustained release delivery system in an amount of about 5%, about 6%, about 7%, about 8%, about 9%, about 10%, about 11%, about 12%, about 13%, about 14%, about 15%, by weight; and the at least one pharmaceutical diluent is present in the sustained release delivery system in an amount of about 55%, about 60%, about 65%, about 70%, about 80%, or about 85% by weight. In one embodiment, the at least one hydrophilic compound is present in the sustained release delivery system in an amount of about 8%, about 12%, or about 20% by weight; the at least one cationic cross-linking agent is present in the sustained release delivery system in an amount of about 10%, about 12%, or about 14% by weight; and the at least one pharmaceutical diluent is present in the sustained release delivery system in an amount of about 40%, about 60%, or about 70% by weight.

In one embodiment, the sustained release delivery system includes about 0.5% to about 80% locust bean gum, about 5% to about 80% xanthan gum, about 20% to about 80% mannitol and about 0.5% to 80% calcium sulfate dihydrate. In one embodiment, the sustained release delivery system includes about 12% to about 47% locust bean gum, about 8% to about 31% xanthan gum, about 20% to about 78% mannitol and about 0.5% to 25% calcium sulfate dihydrate. In one embodiment, the sustained release delivery system includes about 15% to about 25% locust bean gum, about 10% to about 20% xanthan gum, about 50% to about 85% mannitol and about 5% to 15% calcium sulfate dihydrate. In one embodiment, the sustained release delivery system includes about 18% locust bean gum, about 12% xanthan gum, about 60% mannitol and about 10% calcium sulfate dihydrate. In one embodiment, the sustained release delivery system includes about 12% locust bean gum, about 8% xanthan gum, about 70% mannitol and about 10% calcium sulfate dihydrate. In one embodiment, the sustained release delivery system includes about 20% locust bean gum, about 30% xanthan gum, about 40% mannitol and about 10% calcium sulfate dihydrate. In one embodiment, the sustained release delivery system includes about 30% locust bean gum, about 20% xanthan gum, about 40% mannitol and about 10% calcium sulfate dihydrate. In one embodiment, the sustained release delivery system includes about 42% locust bean gum, about 28% xanthan gum, about 20% mannitol and about 10% calcium sulfate dihydrate.

Two properties of the components of this sustained release system (e.g., the at least one hydrophilic compound and the at least one cross-linking agent; or the at least one hydrophilic compound and at least one cationic cross-linking compound) are that it forms a gel matrix upon exposure to liquids are fast hydration of the compounds/agents and the ability to form a gel matrix having a high gel strength. These two properties, which are needed to achieve a slow release gel matrix, are maximized by the particular combination of compounds (e.g., the at least one hydrophilic compound and the at least one cross-linking agent; or the at least one hydrophilic compound and the at least one cationic cross-linking compound). For example, hydrophilic compounds (e.g., xanthan gum) have excellent water-wicking properties that provide fast hydration. The combination of hydrophilic compounds with materials that are capable of cross-linking the rigid helical ordered structure of the hydrophilic compound (e.g., cross-linking agents and/or cationic cross-linking compounds) thereby acts synergistically to provide a higher than expected viscosity (i.e., high gel strength) of the gel matrix.

In some embodiments, the sustained release compositions are further admixed with one or more wetting agents (e.g., polyethoxylated castor oil, polyethoxylated hydrogenated castor oil, polyethoxylated fatty acid from castor oil, polyethoxylated fatty acid from hydrogenated castor oil) one or more lubricants (e.g., magnesium stearate, sodium stearyl fumarate, and the like), one or more buffering agents, one or more colorants, and/or other conventional ingredients.

In some embodiments, compositions employed in the present methods can contain additional pharmaceutical excipients. For example, in some embodiments, fumaric acid can be added to the formulations described herein.

In other embodiments, a non-functional coating, e.g., Opadry® can be added to the compositions described herein.

In some embodiments, the compositions described herein further include a second hydrophilic compound. In some embodiments, the second hydrophilic compound is a cellulose ether. In some embodiments, the second hydrophilic compound is a hydroxyalkyl cellulose or a carboxyalkyl cellulose. In some embodiments, the second hydrophilic compound is a hydroxyethyl cellulose, a hydroxypropyl cellulose, a hydroxypropylmethyl-cellulose, a carboxy methylcellulose, or a mixture thereof. In some embodiments, the second hydrophilic is an ethyl cellulose or wax (e.g., including without limitation cetyl alcohol, stearyl alcohol, white wax, or carnauba wax). The second hydrophilic compound is present in the formulation in an amount ranging from about 5% to about 45%, about 5% to about 25%, about 10% to about 20%, or 12% to about 18% by weight. In some embodiments, the second hydrophilic compound is present in the formulation in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, or about 45%.

In some embodiments, the weight ratio of the second hydrophilic compound to the nalbuphine or pharmaceutically acceptable salt, solvate or ester ranges from about 5:1 to about 1:5, about 4:1 to about 1:4, about 3:1 to about 1:3, about 2:1 to about 1:2, about 1:1 to about 1:3, or about 1:1 to about 1:2. In some embodiments, the weight ratio of the second hydrophilic compound to the nalbuphine or pharmaceutically acceptable salt, solvate or ester is about 5:1, about 4:1, about 3:1, about 2:1, about 1:1, about 1:2, about 1:3, about 1:4, or about 1:5.

In some embodiments, the weight ratio of the second hydrophilic compound to the sustained release delivery system ranges from about 10:1 to about 1:10, about 8:1 to about 1:8, about 6:1 to about 1:6, about 4:1 to about 1:4, about 2:1 to about 1:3, about 1:1 to about 1:10, about 1:1 to about 1:6, or about 1:2 to about 1:6. In some embodiments, the weight ratio of the second hydrophilic compound to the sustained release delivery system is about 10:1, about 8:1, about 6:1, about 4:1, about 2:1, about 1:1, about 1:1.5, about 1:2, about 1:2.5, about 1:3, about 1:4, about 1:5, about 1:6, about 1:7, about 1:8, about 1:9 or about 1:10.

In some embodiments, the oral sustained release solid dosage formulations including from about 1 mg to 200 mg nalbuphine hydrochloride and about 10 mg to about 420 mg of a sustained release delivery system. In these embodiments, the sustained release delivery system includes about 12% to about 42% locust bean gum; about 8.0% to about 28% xanthan gum; about 20% to about 70% mannitol; and about 5% to about 20% calcium sulfate dihydrate. In some embodiments, the present methods can employ oral sustained release solid dosage formulations including from about 5 mg to about 80 mg nalbuphine hydrochloride and about 80 mg to about 360 mg of a sustained release delivery system. In some embodiments, the present methods can employ oral sustained release solid dosage formulations including from about 50 mg to about 150 mg nalbuphine hydrochloride and about 100 mg to about 300 mg of a sustained release delivery system.

In some embodiments, the present methods employ oral sustained release solid dosage formulations including about 15 mg nalbuphine hydrochloride, and from about 25 mg to about 225 mg, for example about 195 mg, of a sustained release delivery system. In these embodiments, the sustained release delivery system includes about 14% locust bean gum; about 9% xanthan gum; about 47% mannitol; and about 8% calcium sulfate dihydrate.

In some embodiments, the present methods employ oral sustained release solid dosage formulations including about 30 mg nalbuphine hydrochloride, and from about 25 mg to about 225 mg, for example about 180 mg, of a sustained release delivery system. In these embodiments, the sustained release delivery system includes about 18% locust bean gum; about 12% xanthan gum; about 60% mannitol; and about 10% calcium sulfate dihydrate.

In some embodiments, the present methods employ oral sustained release solid dosage formulations including about 60 mg nalbuphine hydrochloride, and from about 25 mg to about 225 mg, for example about 120 mg, of a sustained release delivery system. In these embodiments, the sustained release delivery system includes about 10% locust bean gum; about 12% xanthan gum; about 60% mannitol; and about 10% calcium sulfate dihydrate. In some embodiments, the present methods employ oral sustained release solid dosage formulations including from about 5 mg to about 80 mg nalbuphine hydrochloride and about 80 mg to about 360 mg of a sustained release delivery system.

In some embodiments, the present methods employ oral sustained release solid dosage formulations including about 120 mg nalbuphine hydrochloride, and from about 25 mg to about 250 mg, for example about 240 mg, of a sustained release delivery system. In these embodiments, the sustained release delivery system includes about 18% locust bean gum; about 12% xanthan gum; about 60% mannitol; and about 10% calcium sulfate dihydrate.

In some embodiments, the present methods employ oral sustained release solid dosage formulations including about 30 mg nalbuphine hydrochloride, and from about 25 mg to about 350 mg, for example about 270 mg or about 360 mg, of a sustained release delivery system. In these embodiments, the sustained release delivery system includes about 18% locust bean gum; about 12% xanthan gum; about 60% mannitol; and about 10% calcium sulfate dihydrate.

In some embodiments, the present methods employ oral sustained release solid dosage formulations including about 45 to about 60 mg nalbuphine hydrochloride and from about 100 mg to about 200 mg of a sustained release delivery system. In these embodiments, the sustained release delivery system includes about 15% to about 25% locust bean gum; about 10% to about 20% xanthan gum; about 50% to about 85% mannitol; and about 5% to about 15% calcium sulfate dihydrate.

In some embodiments, the present methods employ oral sustained release solid dosage formulations including about 30 mg nalbuphine hydrochloride, about 32.4 mg locust bean gum; about 21.6 mg xanthan gum; about 108 mg mannitol; about 18 mg calcium sulfate dihydrate, about 35 mg hydroxypropylcellulose, and about 1.9 mg magnesium stearate.

In some embodiments, the present methods employ oral sustained release solid dosage formulations including about 29.8 mg nalbuphine hydrochloride, about 32.2 mg locust bean gum; about 21.4 mg xanthan gum; about 107 mg mannitol; about 18 mg calcium sulfate dihydrate, about 35 mg hydroxypropylcellulose, and about 1.9 mg magnesium stearate.

In some embodiments, the present methods employ oral sustained release solid dosage formulations including about 60 mg nalbuphine hydrochloride, about 21.6 mg locust bean gum; about 14.4 mg xanthan gum; about 72 mg mannitol; about 12 mg calcium sulfate dihydrate, about 30 mg hydroxypropylcellulose, and about 1.6 mg magnesium stearate.

In some embodiments, the present methods employ oral sustained release solid dosage formulations including about 59.5 mg nalbuphine hydrochloride, about 21.4 mg locust bean gum; about 14.3 mg xanthan gum; about 71 mg mannitol; about 12 mg calcium sulfate dihydrate, about 30 mg hydroxypropylcellulose, and about 1.6 mg magnesium stearate.

In some embodiments, the present methods employ oral sustained release solid dosage formulations including about 120 mg nalbuphine hydrochloride, about 43.2 mg locust bean gum; about 28.8 mg xanthan gum; about 144 mg mannitol; about 24 mg calcium sulfate dihydrate, about 60 mg hydroxypropylcellulose, and about 3.2 mg magnesium stearate.

In some embodiments, the present methods employ oral sustained release solid dosage formulations including about 119.0 mg nalbuphine hydrochloride, about 42.9 mg locust bean gum; about 25.6 mg xanthan gum; about 143 mg mannitol; about 24 mg calcium sulfate dihydrate, about 60 mg hydroxypropylcellulose, and about 3 mg magnesium stearate.

In some embodiments, the present methods employ oral sustained release solid dosage formulations including about 180 mg nalbuphine hydrochloride, about 64.8 mg locust bean gum; about 43.2 mg xanthan gum; about 216 mg mannitol; about 36 mg calcium sulfate dihydrate, about 90 mg hydroxypropylcellulose, about 5 mg magnesium stearate, and about 25 mg fumaric acid.

In some embodiments, the present methods employ oral sustained release solid dosage formulations including about 180 mg nalbuphine hydrochloride, about 48.6 mg locust bean gum; about 32.4 mg xanthan gum; about 162 mg mannitol; about 27 mg calcium sulfate dihydrate, about 60 mg hydroxypropylcellulose, about 4 mg magnesium stearate, and about 25 mg fumaric acid.

In some embodiments, the present methods employ oral sustained release solid dosage formulations including about 30 mg nalbuphine hydrochloride, about 32.4 mg locust bean gum; about 21.6 mg xanthan gum; about 108 mg mannitol; about 18 mg calcium sulfate dihydrate, about 35 mg hydroxypropylcellulose, about 1.9 mg magnesium stearate, and about 7.4 mg Opadry II White.

In some embodiments, the present methods employ oral sustained release solid dosage formulations including about 178.5 mg nalbuphine hydrochloride, about 48.2 mg locust bean gum; about 32.2 mg xanthan gum; about 161 mg mannitol; about 27 mg calcium sulfate dihydrate, about 60 mg hydroxypropylcellulose, about 4 mg magnesium stearate, and about 25 mg fumaric acid.

The sustained release formulations of nalbuphine are orally administrable solid dosage formulations. Nonlimiting examples of oral solid dosage formulations include tablets, capsules including a plurality of granules, sublingual tablets, powders, granules, syrups, and buccal dosage forms or devices (e.g., buccal patches, tablets, etc.). In some embodiments, tablets have an enteric coating or a hydrophilic coating.

The sustained release delivery system is prepared by dry granulation or wet granulation, before the nalbuphine or pharmaceutically acceptable salt, solvate or ester thereof is added, although the components can be held together by an agglomeration technique to produce an acceptable product. In the wet granulation technique, the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules of the sustained release delivery system. Thereafter, the sustained release delivery system is mixed in the desired amounts with the nalbuphine or the pharmaceutically acceptable salt, solvate or ester thereof and, optionally, one or more wetting agents, one or more lubricants, one or more buffering agents, one or more coloring agents, one or more second hydrophilic compounds, or other conventional ingredients, to produce a granulated composition. The sustained release delivery system and the nalbuphine can be blended with, for example, a high shear mixer. The nalbuphine is preferably finely and homogeneously dispersed in the sustained release delivery system. The granulated composition, in an amount sufficient to make a uniform batch of tablets, is subjected to tableting in a conventional production scale tableting machine at typical compression pressures, i.e., about 2,000-16,000 psi. In some embodiments, the mixture should not be compressed to a point where there is subsequent difficulty with hydration upon exposure to liquids.

In some embodiments, the nalbuphine formulation is prepared by dry granulation or wet granulation. The components of the sustained release delivery system are added, along with the nalbuphine or a pharmaceutically acceptable salt, solvate or ester thereof. Alternatively, all of the components can be held together by an agglomeration technique to produce an acceptable product. In the wet granulation technique, nalbuphine or pharmaceutically salt, solvate or ester thereof and the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules. Optionally, one or more wetting agents, one or more lubricants, one or more buffering agents, one or more coloring agents, one or more second hydrophilic compounds, or other conventional ingredients, are also added to the granulation. The granulated composition, in an amount sufficient to make a uniform batch of tablets, is subjected to tableting in a conventional production scale tableting machine at typical compression pressures, i.e., about 2,000-16,000 psi. In some embodiments, the mixture should not be compressed to a point where there is subsequent difficulty with hydration upon exposure to liquids.

The average particle size of the granulated composition is from about 50 μm to about 400 μm by weight. In some embodiments, the average particle size by weight is from about 185 μm to about 265 The average density of the granulated composition is from about 0.3 g/mL to about 0.8 g/mL. In some embodiments, the average density is from about 0.5 g/mL to about 0.7 g/mL. The tablets formed from the granulations are generally from about 4 Kp to about 22 Kp hardness. The average flow of the granulations is from about 25 to about 40 g/sec.

In some embodiments, the present methods can employ a multilayer solid dosage form, in which the layers are formulated to release the nalbuphine hydrochloride at different rates. For example, in one embodiment, the second layer is an extended release layer that includes nalbuphine or a pharmaceutically acceptable salt, solvate or ester thereof and a sustained release delivery system designed to release the nalbuphine or the pharmaceutically acceptable salt, solvate or ester thereof at a controlled rate so that therapeutically effective blood levels are maintained over an extended period of time (e.g., from about 8 to about 12 hours). The first layer is an immediate release layer that includes a formulation of nalbuphine or a pharmaceutically acceptable salt, solvate or ester thereof designed to release the nalbuphine or the pharmaceutically acceptable salt, solvate or ester thereof at a rate that is faster than the rate of the second layer to achieve a therapeutically effective blood level in an immediate period of time (e.g., from about 1 to about 2 hours). In some embodiments, the first layer includes a sustained release delivery system. In some embodiments, the first layer does not include a sustained release delivery system.

In some embodiments, the weight ratio of the second layer to the first layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2. In one embodiment, the weight ratio of the second layer to the first layer is about 5:1 to about 1:5. In a further embodiment, the weight ratio of the second layer to the first layer is about 1:1 to about 1:2. In some embodiments, the weight ratio of the second layer to the first layer is about 1:1, about 1:1.2, about 1:1.4, about 1:1.6, about 1:1.8, or about 1:2. In one embodiment, the weight ratio of the second layer to the first layer is about 1:2. In one embodiment, the weight ratio of the second layer to the first layer is about 1:1.4. In some embodiments, the weight ratio of the second layer to the first layer is about 3:1, about 2.5:1, about 2:1, about 1.5:1. In one embodiment, the weight ratio of the second layer to the first layer is about 2.5:1.

The sustained release delivery system of the multilayer dosage form includes (i) at least one hydrophilic compound, at least one cross-linking agent, and at least one pharmaceutical diluent; (ii) at least one hydrophilic compound, at least one cross-linking agent, at least one pharmaceutical diluent, and at least one cationic cross-linking agent different from the first cross-linking agent; or (iii) at least one hydrophilic compound, at least one cationic cross-linking compound, and at least one pharmaceutical diluent. In some embodiments, when the first layer includes a sustained release delivery system, the sustained release delivery system of the first layer includes the same components as the sustained release delivery system of the second layer (e.g., both the first and second layers are one of embodiments (i)-(iii), listed above). In other embodiments, the sustained release delivery system of the first layer includes different components as the sustained release delivery system of the second layer (e.g., the first layer is embodiment (i), listed above, while the second layer is embodiment (iii), listed above). It is recognized that the sustained release delivery system of either layer can be one of embodiments (i)-(iii) listed above. Moreover, it is recognized that in some embodiments, the first layer does not include a sustained release delivery system.

The sustained release delivery system is generally present in the second layer (e.g., extended release layer) in an amount ranging from about 10 mg to about 420 mg. In some embodiments, the sustained release delivery system is present in the second layer in an amount ranging from about 110 mg to about 200 mg. In some embodiments, the sustained release delivery system is present in the second layer in an amount ranging from about 110 mg to about 150 mg. In some embodiments, the sustained release delivery system is present in the second layer in an amount ranging from about 90 mg to about 150 mg. In some embodiments, the sustained release delivery system is present in the second layer in an amount of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In one embodiment, the sustained release delivery system is present in the second layer in an amount of about 123 mg. In one embodiment, the sustained release delivery system is present in the second layer in an amount of about 101 mg. In one embodiment, the sustained release delivery system is present in the second layer in an amount of about 92 mg. In another embodiment, the sustained release delivery system is present in the second layer in an amount of about 112.5 mg. In one embodiment, the sustained release delivery system is present in the second layer in an amount of about 135 mg. In one embodiment, the sustained release delivery system is present in the second layer in an amount of about 150 mg.

Nalbuphine or a pharmaceutically acceptable salt, solvate or ester thereof is generally present in the second layer in an amount ranging from about 15 mg to about 60 mg. In some embodiments, nalbuphine or a pharmaceutically acceptable salt, solvate or ester thereof is present in the second layer in an amount ranging from about 30 mg to about 60 mg. In some embodiments, nalbuphine or a pharmaceutically acceptable salt, solvate or ester thereof is present in the second layer in an amount ranging from about 45 mg to about 60 mg. In one embodiment, nalbuphine or a pharmaceutically acceptable salt, solvate or ester thereof is present in the second layer in an amount of about 15 mg. In one embodiment, nalbuphine or a pharmaceutically acceptable salt, solvate or ester thereof is present in the second layer in an amount of about 30 mg. In one embodiment, nalbuphine or a pharmaceutically acceptable salt, solvate or ester thereof is present in the second layer in an amount of about 45 mg. In one embodiment, nalbuphine or a pharmaceutically acceptable salt, solvate or ester thereof is present in the second layer in an amount of about 15 mg, about 30 mg, about 60 mg, about 90 mg, about 120 mg, or about 180 mg.

In some embodiments, the weight ratio of nalbuphine or a pharmaceutically acceptable salt, solvate or ester thereof to the sustained release delivery system in the second layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, or about 2:1 to about 1:2. In one embodiment, the weight ratio of nalbuphine or a pharmaceutically acceptable salt, solvate or ester thereof to the sustained release delivery system in the second layer is about 1:2 to about 1:4. In one embodiment, the weight ratio of nalbuphine or a pharmaceutically acceptable salt, solvate or ester thereof to the sustained release delivery system in the second layer is about 1:1 to about 1:5. In some embodiments, the weight ratio of nalbuphine or a pharmaceutically acceptable salt, solvate or ester thereof to the sustained release delivery system in the second layer is about 1:1, about 1:1.2, about 1:1.4, about 1:1.6, about 1:1.8, about 1:2, about 1:2.5, about 1:3, or about 1:3.5. In one embodiment, the weight ratio of nalbuphine or a pharmaceutically acceptable salt, solvate or ester thereof to the sustained release delivery system in the second layer is about 1:2.5. In another embodiment, the weight ratio of nalbuphine or a pharmaceutically acceptable salt, solvate or ester thereof to the sustained release delivery system in the second layer is about 1:3.3. In a further embodiment, the weight ratio of nalbuphine or a pharmaceutically acceptable salt, solvate or ester thereof to the sustained release delivery system in the second layer is about 1:3. In yet another embodiment, the ratio of nalbuphine or a pharmaceutically acceptable salt, solvate or ester thereof to the sustained release delivery system in the second layer is about 1:2.

When the sustained release delivery system is present in the first layer (e.g., immediate release layer), it is generally present in an amount ranging from about 0 mg to about 50 mg. In some embodiments, the sustained release delivery system is present in the first layer in an amount ranging from about 5 mg to about 25 mg or from about 5 mg to about 15 mg. In one embodiment, the sustained release delivery system is present in the first layer in an amount of about 3 mg to about 9 mg. In one embodiment, the sustained release delivery system is present in the first layer in an amount of about 4 mg to about 6 mg. In some embodiments, the sustained release delivery system is present in the first layer in an amount of about 2 mg, about 4 mg, about 6 mg, about 8 mg, about 10 mg, about 12 mg, about 14 mg, about 15 mg, about 16 mg, about 18 mg, about 20 mg about 25 mg, about 30 mg, about 35 mg, about 40 mg, about 45 mg or about 50 mg. In one embodiment, the sustained release delivery system is present in the first layer in an amount of about 6 mg.

In some embodiments, nalbuphine or a pharmaceutically acceptable salt, solvate or ester thereof is generally present in the first layer (e.g., immediate release layer) in an amount ranging from about 5 mg to about 180 mg. In some embodiments, nalbuphine or a pharmaceutically acceptable salt, solvate or ester thereof is present in the first layer in an amount ranging from about 5 mg to about 25 mg or from about 10 mg to about 20 mg. In some embodiments, the nalbuphine or a pharmaceutically acceptable salt, solvate or ester thereof is present in the first layer in an amount of about 5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg or about 50 mg. In one embodiment, nalbuphine or a pharmaceutically acceptable salt, solvate or ester thereof is present in the first layer in an amount of about 15 mg, about 30 mg, about 60 mg, about 90 mg, about 120 mg, or about 180 mg.

In some embodiments, when the first layer includes a sustained release delivery system, the ratio of nalbuphine or pharmaceutically acceptable salt, solvate or ester thereof to the sustained release delivery system in the first layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2. In one embodiment, the ratio of nalbuphine or pharmaceutically acceptable salt, solvate or ester thereof to the sustained release delivery system in the first layer is about 2:1 to about 4:1. In some embodiments, the ratio of nalbuphine or pharmaceutically acceptable salt, solvate or ester thereof to the sustained release delivery system in the first layer is about 5:1, about 4.5:1, about 4:1, about 3.5:1, about 3:1, about 2.5:1, about 2:1, about 1.5:1, or about 1:1. In one embodiment, the ratio of nalbuphine or pharmaceutically acceptable salt, solvate or ester thereof to the sustained release delivery system in the first layer is about 2.5:1. In another embodiment, the ratio of nalbuphine or pharmaceutically acceptable salt, solvate or ester thereof to the sustained release delivery system in the first layer is about 3:1.

In some embodiments, the multilayer dosage form further includes a pharmaceutical disintegrant. The disintegrant promotes the dissolution and absorption of nalbuphine or pharmaceutically acceptable salt, solvate or ester thereof from the immediate release layer. Nonlimiting examples of pharmaceutical disintegrants include croscarmellose sodium, starch glycolate, crospovidone, and unmodified starch. In one embodiment, the disintegrant is in the first layer (i.e., the immediate release layer), of the dosage form. The disintegrant is generally present in the layer in an amount of about 1.5 mg to about 4.5 mg. In one embodiment, the disintegrant is present in an amount of about 3 mg. In one embodiment, the disintegrant is present in the layer in an amount of about 2-10% by weight. In one embodiment, the disintegrant is present in the layer in an amount of about 5% by weight. When the layer contains a sustained release delivery system, the weight ratio of the sustained release delivery system to the disintegrant is in a range of about 5:1 to about 1:5. In some embodiments, the ratio of the sustained release delivery system to the disintegrant is in a range of about 1:1 to about 3:1. In other embodiments, the ratio of the sustained release delivery system to the disintegrant is in a range of about 2:1.

In some embodiments, the multilayer tablets are prepared by first preparing the immediate release layer and extended release layer blends separately. The extended release layer is prepared as described above. The wet granulation of the extended release layer is then dried and milled to an appropriate size. Magnesium stearate is added and mixed with the milled granulation. The immediate release layer is prepared by first mixing the nalbuphine or the pharmaceutically acceptable salt, solvate or ester thereof with one or more diluents (e.g., microcrystalline cellulose). This mix is then optionally mixed with one or more disintegrants. The blend is mixed with magnesium stearate. Finally, the immediate release layer blend and the extended release layer blend are compressed into multi-layer (e.g., bi-layer) tablets.

In some embodiments, the chemistry of certain of the components of the formulation, such as the hydrophilic compound (e.g., xanthan gum), is such that the components are considered to be self-buffering agents which are substantially insensitive to the solubility of the nalbuphine and the pH changes along the length of the gastrointestinal tract. Moreover, the chemistry of the components is believed to be similar to certain known muco-adhesive substances, such as polycarbophil. Muco-adhesive properties are desirable for buccal delivery systems. Thus, the sustained release formulation can loosely interact with the mucin in the gastrointestinal tract and thereby provide another mode by which a constant rate of delivery of the nalbuphine is achieved.

The phenomenon discussed above (muco-adhesive properties) is a mechanism by which the sustained release formulations can interact with the mucin and fluids of the gastrointestinal tract and provide a constant rate of delivery of the nalbuphine.

When measured by USP Procedure Drug Release General Chapter <711> Dissolution, (incorporated by reference herein in its entirety), the sustained release formulations employed in the present methods generally exhibit an in vitro dissolution of about 15% to about 50% by weight nalbuphine after 1 hour, about 45% to about 80% by weight nalbuphine after 4 hours, or at least about 80% by weight nalbuphine after 10 hours. In some embodiments, the in vitro and in vivo release characteristics of the sustained release formulations are modified using mixtures of one or more different water insoluble and/or water soluble compounds, using different plasticizers, varying the thickness of the sustained release film, including providing release-modifying compounds in the coating, and/or by providing passageways through the coating. In some embodiments, the dissolution rate is determined using apparatus USP Type 111/250 mL at pH 6.8, 37° C. and 15 dpm. In some embodiments, the dissolution rate is determined using apparatus USP Type 111/250 mL performed in pH change (0-1 hours pH 1.2, after hour 1 pH 4.5, after hour 2 pH 6.8) at 37° C. and 15 dpm.

In some embodiments, the sustained release formulation has an in vitro dissolution of about 50% to about 100% by weight nalbuphine after about 6 hours. In some embodiments, the sustained release formulation has an in vitro dissolution of about 75% to about 100% by weight nalbuphine after about 6 hours. In other embodiments, the sustained release formulation has an in vitro dissolution of about 75% to about 100% by weight nalbuphine from about 6 hours to about 8 hours. In further embodiments, the sustained release formulation has an in vitro dissolution of about 80% to about 100% by weight nalbuphine after about 12 hours. In still other embodiments, the sustained release formulation has an in vitro dissolution of about 80% to about 100% by weight nalbuphine from about 12 hours to about 24 hours. In some embodiments, the sustained release formulation has an in vitro dissolution of about 80% to about 100% after about 8 hours to about 12 hours. In yet other embodiments, the sustained release formulation has an in vitro dissolution of about 15% to about 75% by weight nalbuphine after about 1 hour. In still further embodiments, the sustained release formulation has an in vitro dissolution of about 50% by weight nalbuphine after about 1 hour. In some embodiments, the sustained release formulation has an in vitro dissolution of about 50% by weight nalbuphine after about 1 hour and about 75% to about 100% by weight nalbuphine from about 6 hours to about 8 hours. In some embodiments, the sustained release formulation has an in vitro dissolution of about 50% by weight nalbuphine after about 1 hour and about 75% to about 100% by weight nalbuphine from about 8 hours to about 12 hours. In some embodiments, the sustained release formulation has an in vitro dissolution of about 50% by weight nalbuphine after about 1 hour and about 75% to about 100% by weight nalbuphine from about 12 hours to about 24 hours. In some embodiments, the sustained release formulation has an in vitro dissolution of about 50% by weight nalbuphine after about 1 hour and about 80% to about 100% by weight nalbuphine after about 12 hours.

Where the tablet is a multilayer dosage form having a first extended release layer and a second, immediate release, layer, the sustained release formulation has an in vitro dissolution of about 25% to about 75% by weight nalbuphine after about 1 hour. In some embodiments, the multilayer dosage form has an in vitro dissolution of about 25% by weight nalbuphine after about 1 hour. In some embodiments, the multilayer dosage form has an in vitro dissolution of about 50% by weight nalbuphine after about 1 hour. In some embodiments, the multilayer dosage form has an in vitro dissolution of about 75% to about 100% nalbuphine after about 6-8 hours. In some embodiments, the multilayer dosage form has an in vitro dissolution of about 75% to about 100% nalbuphine after about 8-12 hours. In some embodiments, the multilayer dosage form has an in vitro dissolution of about 75% to about 100% nalbuphine after about 12-24 hours. In some embodiments, the multilayer dosage form has an in vitro dissolution of about 75% to about 100% nalbuphine after about 12 hours.

In some embodiments, when administered orally to patients having either normal or impaired (e.g., reduced) kidney function, the sustained release formulations described herein exhibit the following in vivo characteristics: (a) a peak plasma level of nalbuphine occurs within about 4 hours to about 6 hours, e.g., for patients with uremic pruritus or renal impairment, or about 3 hours to about 5 hours, e.g., for patients without renal impairment after administration; (b) onset of nalbuphine anti-pruritic effect from about 30 minutes of dosing to within about 6 hours of dosing; (c) duration of the nalbuphine anti-pruritic effect is about 2 to about 24 hours; and (d) the relative nalbuphine bioavailability is about 0.5, about 1, about 1.5 or between about 0.5 to about 1.5 compared to an orally administered aqueous solution of nalbuphine. The time of onset for an anti-pruritic effect can depend on at least on dosing and the severity of pruritic symptoms. In some embodiments, the duration of the nalbuphine anti-pruritic effect is at least about 8 hours. In some embodiments, the duration of the nalbuphine anti-pruritic effect is at least about 9 hours. In some embodiments, the duration of the nalbuphine anti-pruritic effect is at least about 10 hours. In some embodiments, the duration of the nalbuphine anti-pruritic effect is at least about 11 hours. In some embodiments, the duration of the nalbuphine anti-pruritic effect is at least about 12 hours. In some embodiments, the duration of nalbuphine anti-pruritic effect is about 6, hours, 8 hours, 10 hours, 12 hours, 15 hours, or 18 hours. In some embodiments, the relative nalbuphine bioavailability is about 0.94 compared to an orally administered aqueous solution of nalbuphine. In some embodiments, the relative nalbuphine bioavailability is about 1.35 compared to an orally administered aqueous solution of nalbuphine.

In some embodiments, the sustained release nalbuphine formulations provide an oral unit dosage form including nalbuphine or a pharmaceutically acceptable salt, solvate or ester thereof. The oral dosage form provides an anti-pruritic effect over a period of at least about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours. In some embodiments, the oral dosage form provides an anti-pruritic effect over a period of about 6-18 hours, about 8-16 hours, about 8-12 hours, about 8 to about 24 hours, about 12 to about 24 hours, about 18 to about 24 hours, or about 8-10 hours. The oral dosage form provides an anti-pruritic effect over a period of about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours.

In one embodiment, the oral dosage form provides an anti-pruritic effect as well as breaking the cycle effect, e.g., the itchy sensation does not return after certain treatment period.

In some embodiments, the oral dosage form provides a blood plasma level of nalbuphine characterized by one or more peaks followed by a plateau region. The plateau region is characterized as having a relatively consistent blood plasma level of nalbuphine (e.g., the blood plasma level of nalbuphine does not consistently increase or decrease from time point to time point). In some embodiments, the plateau region is characterized as having a consistent average blood plasma level of nalbuphine. The plateau region is contrasted with the region following the plateau region, in which the blood plasma level of nalbuphine generally decreases from one time point to the next. In some embodiments, the plateau region has a duration of at least about 1 hour, about 2 hours, about 3 hours, about 4 hours, about 5 hours, about 6 hours, about 7 hours, about 8 hours, about 9 hours, about 10 hours, about 11 hours or about 12 hours. In some embodiments, the plateau region has a duration from about 1 hour to about 12 hours, from about 2 hours to about 10 hours, from about 2 hours to about 8 hours, from about 2 hours to about 7 hours or from about 4 hours to about 10 hours, from about 4 hours to about 8 hours, or from about 4 hours to about 6 hours. In some embodiments, the blood plasma level of nalbuphine at each time point in the plateau region ranges from about 75% to about 125% of the mean blood plasma level in the plateau region. In some embodiments, the blood plasma level of nalbuphine at each time point in the plateau region ranges from about 80% to about 120% of the mean blood plasma level in the plateau region. In some embodiments, the blood plasma level of nalbuphine at each time point in the plateau region ranges from about 85% to about 115% of the mean blood plasma level in the plateau region. In some embodiments, the blood plasma level of nalbuphine at each time point in the plateau region ranges from about 90% to about 110% of the mean blood plasma level in the plateau region.

In some embodiments, the minimum blood plasma level of nalbuphine observed during the plateau region is not more than about 25% below the mean blood plasma level for all time points in the plateau region. In some embodiments, the minimum blood plasma level of nalbuphine observed during the plateau region is not more than about 20% below the mean blood plasma level in the plateau region. In some embodiments, the minimum blood plasma level of nalbuphine observed during the plateau region is not more than about 15% below the mean blood plasma level in the plateau region. In some embodiments, the minimum blood plasma level of nalbuphine observed during the plateau region ranges from about 75% to about 100% of the mean blood plasma level in the plateau region. In some embodiments, the minimum blood plasma level of nalbuphine observed during the plateau region ranges from about 80% to about 100% of the mean blood plasma level in the plateau region. In some embodiments, the minimum blood plasma level of nalbuphine observed during the plateau region ranges from about 85% to about 100% of the mean blood plasma level in the plateau region. In some embodiments, the minimum blood plasma level of nalbuphine observed during the plateau region ranges from about 80% to about 95% of the mean blood plasma level in the plateau region.

Co-Therapy

While the compositions can be administered as the sole active pharmaceutical ingredient or sole active anti-pruritus ingredient in the methods described herein, in other embodiments they can also be used in combination with one or more ingredients which are known to be therapeutically effective against pruritus and/or compliment the effect of anti-pruritus ingredient.

For example, in some embodiments, the present methods can employ nalbuphine or a pharmaceutically acceptable salt, solvate or ester thereof in conjunction with one or more anti-pruritic agents. In some embodiments, additional compounds combined with the anti-pruritic agent, e.g., nalbuphine, or a pharmaceutically acceptable salt, solvate or ester thereof, include antihistamines, anti-inflammatory corticosteroids, topical anti-infectives and antifungals, serotonin antagonists, antibacterials, and antivirals, cytotoxic agents, and counter-irritants/analgesics. Other antipruritic agents include anti-depressants, vitamin D, kappa agonists, irritants such as coal tar derivatives and psoralens, 5-HT3 antagonists such as ondansetron, H2 receptor antagonist such as cimetidine, H1 receptor antagonist such as cetirizine, immunomodulators such as tacrolimus, immunosuppressants such as cyclosporine A, μ-antagonists, capsaicin, cannabinoids, latex extracts from various Croton species found in the Amazon jungle (e.g., Zangrado®), fumarate diesters (e.g., monoethylfumarate and dimethylfumarate) or Nk1 antagonists, etc.

In some embodiments, nalbuphine or a pharmaceutically acceptable salt, solvate or ester thereof is not administered in combination with a second anti-pruritus agent, e.g., co-formulated or administered separately.

In some embodiments, nalbuphine or a pharmaceutically acceptable salt, solvate or ester thereof is administered in conjunction with one or more bile sequestrants. In some embodiments, the one or more bile sequestrants is selected from the group consisting of cholestyramine, colestipol and colesevelam. In some embodiments, the bile sequestrant is cholestyramine.

In some embodiments, nalbuphine or a pharmaceutically acceptable salt, solvate or ester thereof is administered in conjunction with one or more pregnane X receptor agonists. In some embodiments, the pregnane X receptor agonists is rifampicin.

In some embodiments, nalbuphine or a pharmaceutically acceptable salt, solvate or ester thereof is administered in conjunction with one or more serotonin reuptake inhibitors. In some embodiments, the serotonin reuptake inhibitor is sertraline.

Dosing

The invention provides methods for treating pruritus by administering an effective amount of an anti-pruritic agent, i.e., nalbuphine or a pharmaceutically acceptable salt, solvate or ester thereof, to a patient in need thereof. An effective amount is an amount sufficient to eliminate or significantly reduce pruritus symptoms or to alleviate those symptoms (e.g., reduce the symptoms, such as itching, compared to the symptoms present prior to treatment). Formulations employed in the present methods can incorporate the anti-pruritic agent in a sustained release formulation such that the formulation provides therapeutically effective blood plasma levels of nalbuphine for the treatment of pruritus.

According to some embodiments of the present disclosure, administering of nalbuphine or a pharmaceutically acceptable salt, solvate or ester thereof provides statistically significant therapeutic effect. In one embodiment, the statistically significant therapeutic effect is determined based on one or more standards or criteria provided by one or more regulatory agencies in the United States, e.g., FDA or other countries. In another embodiment, the statistically significant therapeutic effect is determined based on results obtained from regulatory agency approved clinical trial set up and/or procedure.

In some embodiments, the statistically significant therapeutic effect is determined based on a patient population of at least 20, 50, 60, 100, 200, 300, 400, 500, 600, 700, 800, 900, 1000 or 2000. In some embodiments, the statistically significant therapeutic effect is determined based on data obtained from randomized and double blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%. In some embodiments, the statistically significant therapeutic effect is determined on approval of Phase III clinical trial of the methods provided by the present disclosure, e.g., by FDA in the US.

In some embodiments, the statistically significant therapeutic effect is determined by a randomized double blind clinical trial of patients treated with nalbuphine or a pharmaceutically acceptable salt, solvate or ester thereof and optionally in combination with standard care. In some embodiment, the statistically significant therapeutic effect is determined by a randomized clinical trial and using Numerical Rating Scale (NRS) as primary efficacy parameter and optionally in combination with any other commonly accepted criteria for pruritus assessment.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

According to the present disclosure, the anti-pruritic agent is administered on a once or twice a day basis to provide effective relief of the symptoms of pruritus associated with liver disease (for example, pruritus associated with primary sclerosing cholangitis, primary biliary cholangitis, etc.). In some embodiments, a total daily dose is about 15 mg, about 30 mg, about 60 mg, about 90 mg, about 120 mg, about 180 mg, about 240 mg, about 360 mg, or about 480 mg.

According to the present disclosure, the anti-pruritic agent is administered on a once or twice a day basis to provide effective relief of the symptoms of pruritus associated with obstructive cholestasis secondary to bile duct obstruction due to non-hepatic tissue disease (for example, pruritus associated with pancreatic cancer, pancreatitis, congenital or acquired biliary strictures, lymph node obstruction such as from lymphomas or bile duct stones). In some embodiments, a total daily dose is about 15 mg, about 30 mg, about 60 mg, about 90 mg, about 120 mg, about 180 mg, about 240 mg, about 360 mg, or about 480 mg is administered.

The dosing embodiments described herein refer to the dose required for the treatment of pruritus associated with liver disease. However, the present disclosure contemplates the doses described herein for the treatment of pruritus associated with obstructive cholestasis secondary to bile duct obstruction due to non-hepatic tissue disease.

In some embodiments, the total daily dose of the anti-pruritic agent can be at least about 15 mg a day for the treatment of pruritus associated with liver disease. In some embodiments, the total daily dose of the anti-pruritic agent can be at least about 30 mg a day for the treatment of pruritus associated with liver disease. In some embodiments, the total daily dose of the anti-pruritic agent can be at least about 60 mg a day for the treatment of pruritus associated with liver disease. In some embodiments, the total daily dose of the anti-pruritic agent can be at least about 90 mg a day for the treatment of pruritus associated with liver disease. In some embodiments, the total daily dose of the anti-pruritic agent can be at least about 120 mg a day for the treatment of pruritus associated with liver disease. In some embodiments, the total daily dose of the anti-pruritic agent can be at least about 180 mg a day for the treatment of pruritus associated with liver disease. In some embodiments, the total daily dose of the anti-pruritic agent can be at least about 240 mg a day for the treatment of pruritus associated with liver disease. In some embodiments, the total daily dose of the anti-pruritic agent can be at least about 360 mg a day for the treatment of pruritus associated with liver disease.

In some embodiments, the total daily dose of the anti-pruritic agent can be about 15 mg a day for the treatment of pruritus associated with liver disease. In some embodiments, the total daily dose of the anti-pruritic agent can be about 30 mg a day for the treatment of pruritus associated with liver disease. In some embodiments, the total daily dose of the anti-pruritic agent can be about 60 mg a day for the treatment of pruritus associated with liver disease. In some embodiments, the total daily dose of the anti-pruritic agent can be about 90 mg a day for the treatment of pruritus associated with liver disease. In some embodiments, the total daily dose of the anti-pruritic agent can be about 120 mg a day for the treatment of pruritus associated with liver disease. In some embodiments, the total daily dose of the anti-pruritic agent can be about 180 mg a day for the treatment of pruritus associated with liver disease. In some embodiments, the total daily dose of the anti-pruritic agent can be about 240 mg a day for the treatment of pruritus associated with liver disease. In some embodiments, the total daily dose of the anti-pruritic agent can be about 360 mg a day for the treatment of pruritus associated with liver disease.

In some embodiments, about 15 mg of the anti-pruritus agent once a day is selected to provide a substantial reduction in itch for patients with pruritus associated with liver disease. In some embodiments, about 15 mg of the anti-pruritus agent twice a day is selected to provide a substantial reduction in itch for patients with pruritus associated with liver disease. In some embodiments, about 30 mg of the anti-pruritus agent once a day is selected to provide a substantial reduction in itch for patients with pruritus associated with liver disease. In some embodiments, about 30 mg of the anti-pruritus agent twice a day is selected to provide a substantial reduction in itch for patients with pruritus associated with liver disease. In some embodiments, about 60 mg of the anti-pruritus agent once a day is selected to provide a substantial reduction in itch for patients with pruritus associated with liver disease. In some embodiments, about 60 mg of the anti-pruritus agent twice a day is selected to provide a substantial reduction in itch for patients with pruritus associated with liver disease. In some embodiments, about 90 mg of the anti-pruritus agent once a day is selected to provide a substantial reduction in itch for patients with pruritus associated with liver disease. In some embodiments, about 90 mg of the anti-pruritus agent twice a day is selected to provide a substantial reduction in itch for patients with pruritus associated with liver disease. In some embodiments, about 120 mg of the anti-pruritus agent once a day is selected to provide a substantial reduction in itch for patients with pruritus associated with liver disease. In some embodiments, about 120 mg of the anti-pruritus agent twice a day is selected to provide a substantial reduction in itch for patients with pruritus associated with liver disease. In some embodiments, about 180 mg of the anti-pruritus agent once a day is selected to provide a substantial reduction in itch for patients with pruritus associated with liver disease. In some embodiments, about 180 mg of the anti-pruritus agent twice a day is selected to provide a substantial reduction in itch for patients with pruritus associated with liver disease. In some embodiments, about 360 mg of the anti-pruritus agent once a day is selected to provide a substantial reduction in itch for patients with pruritus associated with liver disease. In some embodiments, about 480 mg of the anti-pruritus agent once a day is selected to provide a substantial reduction in itch for patients with pruritus associated with liver disease.

In some embodiments, about 15 mg of the anti-pruritus agent once a day is selected to provide a reduction of chronic itch in patients with pruritus associated with liver disease. In some embodiments, about 15 mg of the anti-pruritus agent twice a day is selected to provide a reduction of chronic itch in patients with pruritus associated with liver disease. In some embodiments, about 30 mg of the anti-pruritus agent once a day is selected to provide a reduction of chronic itch in patients with pruritus associated with liver disease. In some embodiments, about 30 mg of the anti-pruritus agent twice a day is selected to provide a reduction of chronic itch in patients with pruritus associated with liver disease. In some embodiments, about 60 mg of the anti-pruritus agent once a day is selected to provide a reduction of chronic itch in patients with pruritus associated with liver disease. In some embodiments, about 60 mg of the anti-pruritus agent twice a day is selected to provide a reduction of chronic itch in patients with pruritus associated with liver disease. In some embodiments, about 90 mg of the anti-pruritus agent once a day is selected to provide a reduction of chronic itch in patients with pruritus associated with liver disease. In some embodiments, about 90 mg of the anti-pruritus agent twice a day is selected to provide a reduction of chronic itch in patients with pruritus associated with liver disease. In some embodiments, about 120 mg of the anti-pruritus agent once a day is selected to provide a reduction of chronic itch in patients with pruritus associated with liver disease. In some embodiments, about 120 mg of the anti-pruritus agent twice a day is selected to provide a reduction of chronic itch in patients with pruritus associated with liver disease. In some embodiments, about 180 mg of the anti-pruritus agent once a day is selected to provide a reduction of chronic itch in patients with pruritus associated with liver disease. In some embodiments, about 180 mg of the anti-pruritus agent twice a day is selected to provide a reduction of chronic itch in patients with pruritus associated with liver disease. In some embodiments, about 360 mg of the anti-pruritus agent once a day is selected to provide a substantial reduction in chronic itch for patients with pruritus associated with liver disease. In some embodiments, about 480 mg of the anti-pruritus agent once a day is selected to provide a substantial reduction in chronic itch for patients with pruritus associated with liver disease.

In some embodiments, the amount of anti-pruritic agent administered to a patient in need thereof is in the form of a pharmaceutically acceptable salt and is expressed in terms of the Equivalent Amount of Nalbuphine Free Base provided to said patient.

In some embodiments, the total daily dose of the Equivalent Amount of Nalbuphine Free Base can be at least about 14 mg a day for the treatment of pruritus associated with liver disease. In some embodiments, the total daily dose of the Equivalent Amount of Nalbuphine Free Base can be at least about 27 mg a day for the treatment of pruritus associated with liver disease. In some embodiments, the total daily dose of the Equivalent Amount of Nalbuphine Free Base can be at least about 54 mg a day for the treatment of pruritus associated with liver disease. In some embodiments, the total daily dose of the Equivalent Amount of Nalbuphine Free Base can be at least about 81 mg a day for the treatment of pruritus associated with liver disease. In some embodiments, the total daily dose of the Equivalent Amount of Nalbuphine Free Base can be at least about 108 mg a day for the treatment of pruritus associated with liver disease. In some embodiments, the total daily dose of the Equivalent Amount of Nalbuphine Free Base can be at least about 162 mg a day for the treatment of pruritus associated with liver disease. In some embodiments, the total daily dose of the Equivalent Amount of Nalbuphine Free Base can be at least about 216 mg a day for the treatment of pruritus associated with liver disease.

In some embodiments, the total daily dose of the Equivalent Amount of Nalbuphine Free Base can be about 14 mg a day for the treatment of pruritus associated with liver disease. In some embodiments, the total daily dose of the Equivalent Amount of Nalbuphine Free Base can be about 27 mg a day for the treatment of pruritus associated with liver disease. In some embodiments, the total daily dose of the Equivalent Amount of Nalbuphine Free Base can be about 54 mg a day for the treatment of pruritus associated with liver disease. In some embodiments, the total daily dose of the Equivalent Amount of Nalbuphine Free Base can be about 81 mg a day for the treatment of pruritus associated with liver disease. In some embodiments, the total daily dose of the Equivalent Amount of Nalbuphine Free Base can be about 108 mg a day for the treatment of pruritus associated with liver disease. In some embodiments, the total daily dose of the Equivalent Amount of Nalbuphine Free Base can be about 162 mg a day for the treatment of pruritus associated with liver disease. In some embodiments, the total daily dose of the Equivalent Amount of Nalbuphine Free Base can be about 216 mg a day for the treatment of pruritus associated with liver disease.

In some embodiments, about 14 mg of the Equivalent Amount of Nalbuphine Free Base once a day is selected to provide a substantial reduction in itch for patients with pruritus associated with liver disease. In some embodiments, about 14 mg of the Equivalent Amount of Nalbuphine Free Base twice a day is selected to provide a substantial reduction in itch for patients with pruritus associated with liver disease. In some embodiments, about 27 mg of the Equivalent Amount of Nalbuphine Free Base once a day is selected to provide a substantial reduction in itch for patients with pruritus associated with liver disease. In some embodiments, about 27 mg of the Equivalent Amount of Nalbuphine Free Base twice a day is selected to provide a substantial reduction in itch for patients with pruritus associated with liver disease. In some embodiments, about 54 mg of the Equivalent Amount of Nalbuphine Free Base once a day is selected to provide a substantial reduction in itch for patients with pruritus associated with liver disease. In some embodiments, about 54 mg of the Equivalent Amount of Nalbuphine Free Base twice a day is selected to provide a substantial reduction in itch for patients with pruritus associated with liver disease. In some embodiments, about 81 mg of the Equivalent Amount of Nalbuphine Free Base once a day is selected to provide a substantial reduction in itch for patients with pruritus associated with liver disease. In some embodiments, about 81 mg of the Equivalent Amount of Nalbuphine Free Base twice a day is selected to provide a substantial reduction in itch for patients with pruritus associated with liver disease. In some embodiments, about 108 mg of the Equivalent Amount of Nalbuphine Free Base once a day is selected to provide a substantial reduction in itch for patients with pruritus associated with liver disease. In some embodiments, about 108 mg of the Equivalent Amount of Nalbuphine Free Base twice a day is selected to provide a substantial reduction in itch for patients with pruritus associated with liver disease. In some embodiments, about 162 mg of the Equivalent Amount of Nalbuphine Free Base once a day is selected to provide a substantial reduction in itch for patients with pruritus associated with liver disease. In some embodiments, about 162 mg of the Equivalent Amount of Nalbuphine Free Base twice a day is selected to provide a substantial reduction in itch for patients with pruritus associated with liver disease. In some embodiments, about 216 mg of the Equivalent Amount of Nalbuphine Free Base once a day is selected to provide a substantial reduction in itch for patients with pruritus associated with liver disease.

In some embodiments, about 14 mg of the Equivalent Amount of Nalbuphine Free Base once a day is selected to provide a reduction of chronic itch in patients with pruritus associated with liver disease. In some embodiments, about 14 mg of the Equivalent Amount of Nalbuphine Free Base twice a day is selected to provide a reduction of chronic itch in patients with pruritus associated with liver disease. In some embodiments, about 27 mg of the Equivalent Amount of Nalbuphine Free Base once a day is selected to provide a reduction of chronic itch in patients with pruritus associated with liver disease. In some embodiments, about 27 mg of the Equivalent Amount of Nalbuphine Free Base twice a day is selected to provide a reduction of chronic itch in patients with pruritus associated with liver disease. In some embodiments, about 54 mg of the Equivalent Amount of Nalbuphine Free Base once a day is selected to provide a reduction of chronic itch in patients with pruritus associated with liver disease. In some embodiments, about 54 mg of the Equivalent Amount of Nalbuphine Free Base twice a day is selected to provide a reduction of chronic itch in patients with pruritus associated with liver disease. In some embodiments, about 81 mg of the Equivalent Amount of Nalbuphine Free Base once a day is selected to provide a reduction of chronic itch in patients with pruritus associated with liver disease. In some embodiments, about 81 mg of the Equivalent Amount of Nalbuphine Free Base twice a day is selected to provide a reduction of chronic itch in patients with pruritus associated with liver disease. In some embodiments, about 108 mg of the Equivalent Amount of Nalbuphine Free Base once a day is selected to provide a reduction of chronic itch in patients with pruritus associated with liver disease. In some embodiments, about 108 mg of the Equivalent Amount of Nalbuphine Free Base twice a day is selected to provide a reduction of chronic itch in patients with pruritus associated with liver disease. In some embodiments, about 162 mg of the Equivalent Amount of Nalbuphine Free Base once a day or is selected to provide a reduction of chronic itch in patients with pruritus associated with liver disease. In some embodiments, about 162 mg of the Equivalent Amount of Nalbuphine Free Base twice a day is selected to provide a reduction of chronic itch in patients with pruritus associated with liver disease.

Reduction of itch in patients with pruritic conditions can be determined by various methods. In some embodiments, the effectiveness of a dosage regimen can be determined by evaluation via a Pruritus Visual Analog Scale (VAS) test, such as the worst-itch VAS. In some embodiments, the effectiveness of a dosage regimen can be determined by evaluation via a worst or average itching intensity Numerical Rating Scale (NRS). In yet some other embodiments, the effectiveness of a dosage regimen can be determined by evaluation via a worst or average itching intensity Numerical Rating Scale (NRS), a Patient Global index scale, a Global Physician index scale, Patient Benefit Index—pruritus version (PBI-P), itchy Verbal Rating Scale (VRS) score, ItchyQoL™ (Emory University; http://emoryott.technologypublisher.com/tech?title=ItchyQol%3a_A_Pruritus-Specific_Quality_of_Life_Instrument) or any combination thereof. In still another embodiment, the effectiveness of a dosage regimen can be determined by evaluation via a worst or average itching intensity NRS as a primary efficacy endpoint in association with secondary efficacy endpoints such as the PROMIS Sleep Disturbance Short Form 8a questionnaire, a PROMIS Item Bank v1.0 Fatigue Short Form 7a Scale, PROMIS Item Bank v1.0 PROMIS Sleep Disturbance—Short Form 8a questionnaire, a Patient-Rated Global Assessment of Treatment scale, a Physician-Rated Global Assessment of Treatment scale, Patient Benefit Index—pruritus version (PBI-P), itchy Verbal Rating Scale (VRS) score, the ItchyQoL™ scale or any combination thereof.

According to some embodiments of the present disclosure, the dosing frequency and dose amount per administration of the anti-pruritus agent are selected to provide therapeutic effects for the treatment of pruritus associated obstructive cholestasis secondary to bile duct obstruction due to non-hepatic tissue disease (for example, pruritus associated with pancreatic cancer, pancreatitis, congenital or acquired biliary strictures, lymph node obstruction such as from lymphomas or bile duct stones).

According to some embodiments of the present disclosure, the dosing frequency and dose amount per administration of the anti-pruritus agent are selected to provide therapeutic effects for the treatment of pruritus associated with liver disease (for example, pruritus associated with primary sclerosing cholangitis, primary biliary cholangitis, etc.).

According to some embodiments of the present disclosure, the dosing frequency and dose amount per administration of the anti-pruritus agent are selected to provide therapeutic effects for the treatment of pruritus associated with liver disease selected from cholestatic liver disease, infectious hepatitis; cirrhotic liver disease, drug-induced liver disease, idiopathic portal hypertension, congenital malformations or genetic diseases affecting liver function, sarcoidosis, primary or metastatic neoplasm involvement of the liver and autoimmune hepatitis-cholangitis (Overlap syndrome).

According to some embodiments of the present disclosure, the dosing frequency and dose amount per administration of the anti-pruritus agent are selected to provide therapeutic effects for the treatment of pruritus associated with liver disease that is refractory to other treatments. In some embodiments, the dosing frequency and dose amount per administration of the anti-pruritus agent are selected to provide therapeutic effects for the treatment of pruritus associated with liver disease that is refractory to treatment with other anti-pruritus agents, refractory to treatment with bile sequestrants or refractory to treatment with rifampicin.

In some embodiments, nalbuphine or a pharmaceutically acceptable salt, solvate or ester thereof is administered on a once-a-day or twice-a-day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks.

In some embodiments, at least about 15 mg or about 15 mg of nalbuphine or a pharmaceutically acceptable salt, solvate or ester thereof is administered on a once-a-day or twice-a-day basis for at least a week. In some embodiments, at least about 30 mg or about 30 mg of nalbuphine or a pharmaceutically acceptable salt, solvate or ester thereof is administered on a once-a-day or twice-a-day basis for at least a week. In some embodiments, at least about 60 mg or about 60 mg of nalbuphine or a pharmaceutically acceptable salt, solvate or ester thereof is administered on a once-a-day or twice-a-day basis for at least a week. In some embodiments, at least about 90 mg or about 90 mg of nalbuphine or a pharmaceutically acceptable salt, solvate or ester thereof is administered on a once-a-day or twice-a-day basis for at least a week. In some embodiments, at least about 120 mg or about 120 mg of nalbuphine or a pharmaceutically acceptable salt, solvate or ester thereof is administered on a once-a-day or twice-a-day basis for at least a week. In some embodiments, at least about 180 mg or about 180 mg of nalbuphine or a pharmaceutically acceptable salt, solvate or ester thereof is administered on a once-a-day or twice-a-day basis for at least a week. In some embodiments, at least about 240 mg or about 240 mg of nalbuphine or a pharmaceutically acceptable salt, solvate or ester thereof is administered on a once-a-day or twice-a-day basis for at least a week. In some embodiments, at least about 360 mg or about 360 mg of nalbuphine or a pharmaceutically acceptable salt, solvate or ester thereof is administered on a once-a-day or twice-a-day basis for at least a week.

According to some embodiments, the substantial reduction in itch provided by the methods of the present disclosure requires treatment for a specified time interval (e.g., at least one week) before the patient experiences substantial reduction of itch (i.e., there is an induction period before the patient experiences a substantial reduction in itch). In some embodiments, after treatment for at least one week, at least two weeks, at least three weeks, at least four weeks, at least five weeks, at least six weeks, at least seven weeks or at least eight weeks, the patient experiences a substantial reduction of itch compared to prior to the treatment. In some embodiments, after treatment for at least one week the patient experiences a substantial reduction of itch compared to prior to the treatment. According to this embodiment, the substantial reduction in itch may be expressed using any of the methods described herein (for example, decline in worst or average itching intensity Numerical Rating Scale value compared to prior to the treatment, improvement in the ItchyQoL™ scale compared to prior to the treatment, etc.).

In some embodiments, after the treatment the patient experiences a substantial reduction of itch that is characterized by at least about a 30% decline in worst or average itching intensity Numerical Rating Scale (NRS) value compared to prior to the treatment. In some embodiments, the reduction of itch is characterized by a decline in NRS value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of itch that is characterized by at least a one point decline in worst or average itching intensity Numerical Rating Scale (NRS) value compared to prior to the treatment. In some embodiments, the reduction of itch is characterized by a decline in worst or average itching intensity NRS value ranging from about one point to about five points, for example, about one point, about two points, about three points, about four points, and about five points compared to prior to the treatment. In some embodiments, the reduction of itch is characterized by a decline in worst or average itching intensity NRS value of about two points. In some embodiments, the reduction of itch is characterized by a decline in worst or average itching intensity NRS value of about three points. In some embodiments, the reduction of itch is characterized by a decline in worst or average itching intensity NRS value of about four points. In some embodiments, the reduction of itch is characterized by a decline in worst or average itching intensity NRS value of about five points.

In some embodiments, after the treatment the patient experiences a substantial reduction of itch that is characterized by at least about a 10% improvement in the ItchyQoL scale compared to prior to the treatment. In some embodiments, the reduction of itch is characterized by an improvement in ItchyQoL scale ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of itch that is characterized by an improvement in sleep that is characterized by at least a one category change in at least one of the 8 questions of the PROMIS Item Bank v1.0 PROMIS Sleep Disturbance—Short Form 8a questionnaire. In some embodiments, the reduction of itch is characterized by an improvement in PROMIS Sleep Disturbance Short Form 8a questionnaire total raw score or any of the respective subscale by one category (one unit), compared to prior to the treatment.

In some embodiments, after the treatment the patient experiences a substantial reduction of itch that is characterized by a reduction of fatigue that is characterized by at least a one category change in at least one of the 7 questions of the PROMIS Item Bank v1.0 Fatigue Short Form 7a questionnaire compared to prior to the treatment. In some embodiments, the reduction of itch is characterized by an improvement in PROMIS Fatigue Short Form 7a questionnaire total raw score or any of the respective subscale by one category (one unit), compared to prior to the treatment.

In some embodiments, after the treatment the patient experiences a substantial reduction of itchy Verbal Rating Scale (VRS) score compared to prior to the treatment. In some embodiments, the reduction of itch is characterized by an improvement in itchy VRS score ranging from at least one category (or unit of change) to about three categories, for example, about one category, about two categories, and about three categories compared to prior to the treatment. In some embodiments, the reduction of itch is characterized by an improvement in itchy VRS score of at least one category (or unit of change). In some embodiments, the reduction of itch is characterized by an improvement in itchy VRS score of at least two categories (or unit of change). In some embodiments, the reduction of itch is characterized by an improvement in itchy VRS score of at least three categories (or unit of change).

In some embodiments, after the treatment the patient experiences a substantial reduction of itch that is characterized by at least about a 10% improvement in Patient Benefit Index—pruritus version (PBI-P) scale compared to prior to the treatment. In some embodiments, the reduction of itch is characterized by an improvement in Patient Benefit Index—pruritus version (PBI-P) scale ranging from about 10% to about 100%, for example, about 10%, about 20%, about 30%, about 40%, about 50%, about 60%, about 70%, about 80%, about 90%, and about 100%, compared to prior to the treatment.

In some embodiments, the daily dose of the anti-pruritic agent is in a once or twice daily dose, and then titrated upward until the patient experiences satisfactory relief from the pruritic condition. The daily dose can be titrated in increments ranging from about 15 mg to about 60 mg (e.g., about 15 mg, about 30 mg or about 60 mg). The daily dose can be titrated in one or more steps. The daily dosage can be titrated by increasing a single daily dosage, or each dose of a twice-daily dosing regimen. The amount a dosage is stepped, where there are multiple titration steps, can be the same, or can be different.

In some embodiments, the titration may be initiated with about 15 mg, about 30 mg or about 60 mg of the anti-pruritic agent once or twice daily. In some embodiments, doses can be adjusted in 30 mg increments every 1 to 4 days. Patients can self-titrate to effect over from about 7 days to about 30 days (for example, from about 12 days to about 20 days) to a dose that provides adequate relief from itch and minimizes adverse reactions. In some embodiments, the titration is conducted for at least about one week, about 2 weeks, about 3 weeks, about 4 weeks or about 5 weeks until a steady state is achieved in the patient.

In some embodiments, patients can be provided initially with 15 mg, 30 mg or 60 mg tablets to self-titrate to effect up to about 60 mg, about 90 mg, about 120 mg, about 180 mg, about 240 mg, about 360 mg, or about 480 mg once or twice a day. In some embodiments, the titration dose is started with about 15 mg or about 30 mg, and then gradually increased to about 60 mg or 120 mg twice a day, e.g., for patients with pruritus associated with liver disease. In some embodiments, the titration dose is started with about 15 mg or about 30 mg, and then gradually increased to about 60 mg or 120 mg once a day, e.g., for patients with pruritus associated with liver disease. In another embodiment, the titration dose is started with about 15 mg or about 30 mg, and then gradually increased to about 120 mg or 240 mg twice a day, e.g., for patients with pruritus associated with liver disease. In another embodiment, the titration dose is started with about 15 mg or about 30 mg, and then gradually increased to about 120 mg or 240 mg once a day, e.g., for patients with pruritus associated with liver disease.

In some embodiments, the anti-pruritic agent is nalbuphine and the titration is conducted for two weeks according to the dose schedule provided in the following table:

| Day | AM dosage (mg) | PM dosage (mg) |
|---|---|---|
| Day 1 | 0 | 30 |
| Day 2 | 0 | 30 |
| Day 3 | 30 | 30 |
| Day 4 | 30 | 30 |
| Day 5 | 30 | 60 |
| Day 6 | 60 | 60 |
| Day 7 | 60 | 60 |
| Day 8 | 60 | 90 |
| Day 9 | 90 | 90 |
| Day 10 | 90 | 90 |
| Day 11 | 90 | 120 |
| Day 12 | 120 | 120 |
| Day 13 | 120 | 120 |
| Day 14 | 120 | 180 |

In some embodiments, the anti-pruritic agent is nalbuphine and the titration is conducted for two weeks according to the dose schedule provided in the following table:

| Day | AM dosage (mg) | PM dosage (mg) |
|---|---|---|
| Day 1 | 0 | 30 |
| Day 2 | 0 | 30 |
| Day 3 | 30 | 30 |
| Day 4 | 30 | 30 |
| Day 5 | 30 | 60 |
| Day 6 | 60 | 60 |
| Day 7 | 60 | 60 |
| Day 8 | 60 | 90 |
| Day 9 | 90 | 90 |
| Day 10 | 90 | 90 |
| Day 11 | 90 | 120 |
| Day 12 | 120 | 120 |
| Day 13 | 120 | 120 |
| Day 14 | 120 | 120 |

In some embodiments, the anti-pruritic agent is nalbuphine and the titration is conducted for seventeen days according to the dose schedule provided in the following table:

| Day | AM dosage (mg) | PM dosage (mg) |
|---|---|---|
| Day 1 | 0 | 30 |
| Day 2 | 0 | 30 |
| Day 3 | 30 | 30 |
| Day 4 | 30 | 30 |
| Day 5 | 30 | 60 |
| Day 6 | 60 | 60 |
| Day 7 | 60 | 60 |
| Day 8 | 60 | 60 |
| Day 9 | 60 | 120 |
| Day 10 | 120 | 120 |
| Day 11 | 120 | 120 |
| Day 12 | 120 | 120 |
| Day 13 | 120 | 120 |
| Day 14 | 120 | 120 |
| Day 15 | 120 | 120 |
| Day 16 | 120 | 180 |
| Day 17 | 180 | 180 |

According to some embodiments of the present disclosure, the methods of the present disclosure provide therapeutically effective blood plasma levels of nalbuphine for treating patients with pruritus associated with liver disease. Blood plasma levels of nalbuphine may be expressed using pharmacokinetic parameters that are known to those skilled in the art, such as steady state plasma levels, AUC, Cmax and Cmin. Blood plasma levels of nalbuphine are described in U.S. Publication Nos. 2014/0171459, 2014/0350042, 2015/0359789, and 2017/0216277, which are incorporated by reference herein in their entirety.

In some embodiments, the present methods provide steady state plasma levels of nalbuphine that correlate to one or more statistically significant therapeutic effects. In some embodiments, the therapeutically effective steady state plasma levels of nalbuphine provided by the methods of the present disclosure range from about 10 ng/mL to about 80 ng/mL, including about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL and about 80 ng/mL, including all ranges there between. In some embodiments, the therapeutically effective steady state plasma levels of nalbuphine is provided by administering a daily dose of nalbuphine or a pharmaceutically acceptable salt or ester is about 360 mg. In further embodiments, the therapeutically effective steady state plasma levels of nalbuphine is provided by administering about 180 mg of nalbuphine or a pharmaceutically acceptable salt or ester thereof twice a day.

In some embodiments, the present methods provide mean steady state AUC₀₋₂₄ₕ (expressed in terms of ng*hr/mL) levels of nalbuphine that correlate to one or more statistically significant therapeutic effects. In some embodiments, the therapeutically effective mean steady state AUC₀₋₂₄ₕ levels of nalbuphine provided by the methods of the present disclosure range from about 200 ng*hr/mL to about 1600 ng*hr/mL, including about 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, and about 1500 ng*hr/mL, including all ranges there between. In some embodiments, the therapeutically effective mean steady state AUC₀₋₂₄ₕ levels of nalbuphine is provided by administering a daily dose of nalbuphine or a pharmaceutically acceptable salt or ester is about 360 mg. In further embodiments, the therapeutically effective mean steady state AUC₀₋₂₄ₕ levels of nalbuphine is provided by administering about 180 mg of nalbuphine or a pharmaceutically acceptable salt or ester thereof twice a day.

In some embodiments, the anti-pruritus agent is nalbuphine, and the metabolites include glucuronides (most likely on the phenol and cyclohexane rings), two hydroxylated nalbuphine metabolites (on the cyclobutane ring) and three ketones (hydroxylation of the cyclobutane ring, followed by oxidation to a carbonyl or followed by ring opening of the cyclobutane ring). In some embodiments, the nalbuphine metabolites include nalbuphine 3-glucuronide or 6-glucuronide. In some other embodiments, the nalbuphine metabolites include triple hydroxylated nalbuphine, mono-hydroxylated nalbuphine, or mono-glucuronidated nalbuphine or a combination thereof. In some embodiments, the one or more metabolites of the anti-pruritus agent do not have detectable anti-pruritus activity. In other embodiments, one or more of the metabolites of the anti-pruritus agent exhibit anti-pruritus activity.

In embodiments wherein one or more metabolites of the anti-pruritus agent exhibit anti-pruritus activity, the dosing regimen of the anti-pruritus agent may be adjusted and/or titrated as described hereinabove depending on the clearance rate of the one or more metabolites exhibiting anti-pruritic activity. Such dosage adjustment and/or titration of the dosage of the anti-pruritic agent can be performed to prevent accumulation of either the anti-pruritic agent and/or one or more metabolites, which can also exhibit anti-pruritic activity, to avoid toxicity effects in a patient treated with the present anti-pruritic agent.

In some embodiments, the anti-pruritus agent is completely metabolized (e.g., about 100% metabolized). In other embodiments, the anti-pruritus agent is not completely metabolized (e.g., less than about 100% metabolized). For example, in some embodiments, the anti-pruritus agent is about 100% metabolized, about 95% metabolized, about 90% metabolized, about 85% metabolized, about 80% metabolized, about 75% metabolized, about 70% metabolized, about 65% metabolized, about 60% metabolized, about 55% metabolized, about 50% metabolized, about 45% metabolized, about 40% metabolized, about 35% metabolized, about 25% metabolized, about 20% metabolized, about 15% metabolized, about 10% metabolized, about 5% metabolized, about 1% metabolized, or about 0% metabolized. In some embodiments, the amount of dialyzable agent can be measured or monitored by the level of accumulation, e.g., blood plasma level of the anti-pruritus agent or one or more of its metabolites.

The embodiments described herein should be understood to be illustrative of the present disclosure, and should not be construed as limiting. On the contrary, the present disclosure embraces alternatives and equivalents thereof, as embodied by the appended claims. Each reference disclosed herein is incorporated by reference herein in its entirety.

The following non-limiting examples illustrate various aspects of the present disclosure.

### EXAMPLES

### Example 1

A 30 mg, 60 mg, 120 or 180 mg extended release (ER) nalbuphine tablet was prepared as follows: Nalbuphine HCl, mannitol, xanthan gum, locust bean gum and calcium sulfate dihydrate were added to a high shear mixer and dried mix at low speed. A granulating solution (water for injection or purified water) was introduced into the mixer at low speed. The wet granulation was granulated at high speed and dried in a fluid bed processor. The dried granules were milled and sized using a conventional mill. The milled granulation was transferred into a diffusion (tumble) mixer. Hydroxypropylcellulose and, when applicable, fumaric acid (180 mg formulations only) were added to the diffusion mixer and blended. Thereafter, magnesium stearate was added to the diffusion mixer and blended. The final blend was compressed using a rotary tablet press. Tablets may be coated with a non-functional Opadry white coating.

TABLE 1
30 mg, 60 mg, 120 mg and 180 mg Extended
Release Nalbuphine Tablet

| Ingredient | mg/tablet |
|---|---|
| Nalbuphine HCl | 29.8 |
| Mannitol | 107.3 |
| Hydroxypropylcellulose | 34.7 |
| Locust bean gum | 32.2 |
| Xanthan gum | 21.4 |
| Calcium sulfate dehydrate | 17.9 |
| Magnesium stearate | 1.9 |
| Water for injection or Purified water | QS |
| Total: | 245.1 |
| Nalbuphine HCl | 59.5 |
| Mannitol | 71.5 |
| Hydroxypropylcellulose | 29.8 |
| Locust bean gum | 21.4 |
| Xanthan gum | 14.3 |
| Calcium sulfate dehydrate | 11.9 |
| Magnesium stearate | 1.6 |
| Water for injection or Purified water | QS |
| Total: | 210.0 |
| Nalbuphine HCl | 119.0 |
| Mannitol | 143.0 |
| Hydroxypropylcellulose | 59.6 |
| Locust bean gum | 42.9 |
| Xanthan gum | 28.6 |
| Calcium sulfate dehydrate | 23.8 |
| Magnesium stearate | 3.2 |
| Water for injection or Purified water | QS |
| Total: | 432.6 |
| Nalbuphine HCl | 178.5 |
| Mannitol | 160.8 |
| Hydroxypropylcellulose | 59.6 |
| Locust bean gum | 48.2 |
| Xanthan gum | 32.2 |
| Calcium sulfate dehydrate | 26.8 |
| Magnesium stearate | 4.0 |
| Fumaric acid | 24.8 |
| Water for injection or Purified water | QS |
| Total: | 246.9 |

The tablets were coated with a non-functional coat (Opadry II White).

TABLE 2
Nalbuphine HCl ER Tablets, 30 mg,
60 mg, or 180 mg Compositions

| Component | Tablet (mg/tablet) |
|---|---|
| Nalbuphine HCl | 30.0 |
| Mannitol | 108.0 |
| Hydroxypropylcellulose | 35.0 |
| Locust bean gum | 32.4 |
| Xanthan gum | 21.6 |
| Calcium sulfate dihydrate | 18.0 |
| Magnesium stearate | 1.9 |
| Opadry II White | 7.4 |
| Sterile water for irrigation | QS |
| Total | 254.3 |
| Nalbuphine HCl | 60.0 |
| Mannitol | 72.0 |
| Hydroxypropylcellulose | 30.0 |
| Locust bean gum | 21.6 |
| Xanthan gum | 14.4 |
| Calcium sulfate dihydrate | 12.0 |
| Magnesium stearate | 1.6 |
| Opadry II White | 6.355 |
| Sterile water for irrigation | QS |
| Total | 218 |
| Nalbuphine HCl | 180 |
| Mannitol | 160.8 |
| Hydroxypropylcellulose | 59.6 |
| Locust bean gum | 48.2 |
| Fumaric acid | 24.8 |
| Xanthan gum | 32.2 |
| Calcium sulfate dihydrate | 26.8 |
| Magnesium stearate | 4.0 |
| Sterile water for irrigation | QS |
| Total | 534.9 |

### Example 2

Healthy and liver-impaired patients will be treated with Nalbuphine extended release (ER) tablets, prepared according to the formulations described herein, to study the effect of hepatic impairment on the pharmacokinetics at steady state as a function of dose. Dose and dose frequencies will be evaluated in order to select a regimen that is suitable for subjects with impaired hepatic function. From the results of the study, oral nalbuphine will be assessed for its potential to reduce liver itch in a dose-dependent manner.

Study Design

The study will be an open-label, multiple escalating dose study comprised of 2 cohorts that each receive Nalbuphine ER tablets of the present disclosure. Cohort 1 will consist of subjects with impaired hepatic function divided into three groups with some subjects in each of the mild Child-Pugh category (Group 1), the moderate Child-Pugh category, and the severe Child-Pugh category. Cohort 2 will consist of healthy subjects.

Dosing:

Subjects will receive a single 27 mg dose of a Nalbuphine ER tablet on the morning of Day 1. Doses will be subsequently escalated for each subject to 27 mg, 54 mg, 108 mg, and 162 mg (Equivalent Amount of Nalbuphine Free Base) twice daily (BID) over 13 days. On the last treatment day (Day 14), subjects will receive a single 162 mg dose in the morning. Subjects will remain at each dose level for 2-3 days, with a minimum of 4-6 consecutive doses provided (see Table 3). Dose escalation will be predicated on tolerability of the prior dose, and can be halted based on adverse events. Dosing of subjects in Cohort 1 groups may be staggered to allow for an interim PK analysis.

Blood and urine will be obtained during each treatment period at designated times for PK and other analyses (see below). Standard safety assessments will be measured during each treatment period.

Pharmacokinetic (PK) Assessments

PK parameters (e.g., Cₘₐₓ, Tₘₐₓ, T_{1/2}, AUC, relative bioavailability, etc.) for healthy patients and patients with hepatic impairment will be compared to assess the suitability of Nalbuphine ER tablets for the treatment of liver itch. Data will be obtained from the blood plasma samples collected from each cohort according to the schedule provided. Graphical representations of the data, such as of nalbuphine plasma concentration versus time, can be prepared once the analysis is completed.

Plasma samples will be analyzed to determine nalbuphine concentrations using a validated assay method. Pharmacokinetic variables (including but not limited to Cₘₐₓ, Tₘₐₓ and AUC₍₀₋ₗₐₛₜ₎) will be calculated using non-compartmental analysis. PK parameters for nalbuphine will be derived from the plasma concentration data using non-compartmental analysis with WinNonlin Professional software (version 5.2 or higher).

Protocol:

Blood:

Blood from the patients of each cohort will be collected in K₂EDTA tubes. The plasma fraction will be separated by centrifugation and stored frozen until analysis. Blood samples will be collected on Days 1 and 13 at the following timepoints: 0 h (prior to the morning dose), 1 h, 2 h, 3 h, 4 h, 5 h, 6 h, 7 h, 9 h, 12 h, 18 h, 24 h, and at 30 h, 36 h, 48 h, and 72 h after the morning dose (Day 13 only), for the purpose of calculating terminal T_{1/2}.

Trough level blood samples will be collected on Days 2, 4, 7, 10, and 12, prior to the morning and evening dose administrations. The trough blood sample prior to morning dose on Day 2 serves as a 24-hour post-dose sample for the Day 1 PK profile. Other samples may be collected at the discretion of the investigator.

Urine:

Urine may be collected pre-dose (−2 h to 0 h) on Day 1 at intervals of 0-12 h, 12-24 h, and 24-48 h post-morning dose following last dose of nalbuphine. Urine volume and time of collection will be recorded and duplicate aliquots will be transferred into freezing tubes and stored frozen until analysis. Urine samples will be analyzed to determine nalbuphine concentrations using a validated assay method. Pooling of urine across patients may be allowed if volumes are not sufficient to allow individual determination.

TABLE 3
Representative dose escalation and schedule for
patients treated with Nalbuphine ER tablets.

| | | | | | Target |
|---|---|---|---|---|---|
| | | | | Total Daily | Plasma Sampling |
| Day | Dose (mg) | | Frequency^{b} | Dose (mg/kg) | Time Analysis |
| 1 | 27 | 0 | QD | 27 | 0 h, 1 h, 2 h, 3 h, 4 h, 5 h, |
| | | | | | 6 h, 7 h, 9 h, 12 h, 18 h after |
| | | | | | morning dose |
| 2 | 27 | 27 | BID | 54 | Prior to morning^{c}/evening dose; |
| 3 | 27 | 27 | BID | 54 | |
| 4 | 27 | 54 | BID | 54 | Prior to morning^{c}/evening dose; |
| 5 | 54 | 54 | BID | 108 | |
| 6 | 54 | 54 | BID | 108 | |
| 7 | 54 | 108 | BID | 108 | Prior to morning/evening dose |
| 8 | 108 | 108 | BID | 216 | |
| 9 | 108 | 108 | BID | 216 | |
| 10 | 108 | 162 | BID | 216 | Prior to morning/evening dose |
| 11 | 162 | 162 | BID | 324 | |
| 12 | 162 | 162 | BID | 324 | Prior to morning/evening dose |
| 13 | 162 | 0 | QD | 162 | 0 h, 1 h, 2 h, 3 h, 4 h, 5 h, |
| | | | | | 6 h, 7 h, 9 h, 12 h, 18 h |
| 14 | 0 | 0 | | 0 | 24, 30, 36 (post Day 13 dose ) |

| | | | |
|---|---|---|---|
| 15 | 0 | 0 | 48 (post Day 13 dose) |
| 16 | 0 | Discharge ~48 h to 72 | 48 (post Day 13 dose) |
| | | after last dose | |

^{a} Subjects will remain at each dose level for at least two days and for a minimum of four consecutive doses
^{b}BID constitutes a morning and an evening dose
^{c}Morning dose constitutes Day 2 trough blood sample
^{d} Morning dose constitutes Day 3 trough blood sample
^{e} Morning dose constitutes Day 4 trough blood sample

Pharmacodynamic Assessment

A numerical rating scale (NRS) will be used to determine the itch severity experienced by subjects at set timepoints on a daily basis, potentially twice a day—once within an hour of completing their morning and evening meals.

Safety Assessments/Monitoring

Adverse events (AEs) will be monitored throughout the duration of the study.

To monitor for possible adverse events, sitting blood pressure, heart rate, body temperature, clinical laboratory tests (hematology, chemistry, and urinalysis) and respiration rate will be monitored and physical examination and 12-lead ECG will be conducted during the study.

Statistical Analysis

For all subjects who receive at least one dose of nalbuphine (Safety Population), treatment-emergent AEs (TEAEs) will be summarized by each nalbuphine dose level (per period) by system organ class and preferred term, by maximum severity, as well as by relationship to treatment.

All AEs will be presented in a listing ordered by subject and onset day. Serious adverse events (SAEs), AEs leading to study discontinuation, and AEs with an outcome of death will each be presented in separate listings as necessary.

Vital signs, clinical safety laboratory tests, and ECG interval data will be summarized descriptively (sample size (N), mean, median, standard deviation, minimum, and maximum) by dose. Vital signs, safety laboratory parameters, ECGs, and physical examination findings will be listed by cohort, subject, treatment, and study day within the treatment period. Listings will include scheduled, unscheduled, and repeat evaluations. The listing of vital signs will include the change from pre-dose of the current treatment period. All clinically significant changes in vital signs, safety laboratory parameters, and physical examination findings will be listed by cohort, subject, treatment, and study day within the treatment period.

Pharmacokinetic parameters will be generated for all subjects with nalbuphine plasma concentration data above the lowest level of quantification (LLOQ) for any treatment, and information for the PK Evaluable Population will be provided in the data listings. Pharmacokinetic parameters from urine will also be generated and presented in the data listings.

The PK Evaluable Population will be defined to include all subjects who received nalbuphine and have evaluable PK data.

All nalbuphine PK results of the PK Evaluable Population will be summarized using appropriate descriptive statistics, including the number of subjects (N), mean, standard deviation, minimum and maximum values and CV %. Geometric mean and geometric CV % values will also be derived for nalbuphine Cmax, and AUC₍₀₋ₗₐₛₜ₎ parameters. Descriptive statistics for Tmax will be summarized using mean, median, minimum and maximum values only, along with the number of subjects (N).

Within each group, dose proportionality will be assessed by a visual assessment of the individual and mean nalbuphine PK parameters. For all subjects who receive at least 1 dose of study drug (Safety Population), AEs will be analyzed and summarized descriptively by total number of AEs for each treatment, and the number and frequency of subjects reporting any AEs by body system and treatment. AEs will be categorized by all treatment-emergent AEs, all severe AEs, treatment-related AEs, and severe treatment-related AEs. Vital signs, clinical safety laboratory tests, and ECG interval data will be analyzed and summarized descriptively (sample size (n), mean, median, standard deviation, minimum, and maximum) by treatment. All clinically significant changes in vital signs, safety laboratory parameters, physical examination findings, and ECG abnormalities will be listed by treatment group, subject, period, and study day within the treatment period.

### Example 3

Liver-impaired patients will be treated with Nalbuphine extended release (ER) tablets, prepared according to the formulations described herein, in a two-part study design. The first part will be a single-ascending dose study followed by a multiple-escalating dose study to determine the effect of hepatic impairment on the pharmacokinetics at steady state as a function of dose. Healthy subjects will receive a single dose of drug at the highest dose studied in the liver-impaired subjects in order to make relative comparison to the PK aspects of Nalbuphine extended release (ER).

Study Design

The study will be a two-part open-label, single ascending dose and multiple escalating dose study comprised of six cohorts that each receive Nalbuphine ER tablets of the present disclosure. Part 1 will be a single ascending dose (SAD) arm and will consist of five cohorts. Cohorts 1-4 will consist of subjects with impaired hepatic function divided into three groups with some subjects in each of the mild Child-Pugh category (Group 1), the moderate Child-Pugh category (Group 2), and the severe Child-Pugh category (Group 3). Cohort 5 will consist of healthy control subjects who have been appropriately age-, body mass index (BMI), and gender-matched to subjects with mild and moderate hepatic impairment from Cohorts 1 to 4. Cohort 6 will consist of subjects with impaired hepatic function divided into two groups with some subjects in each of the mild Child-Pugh category (Group 1) and the moderate Child-Pugh category (Group 2).

Part 2 will be a multiple ascending dose cohort and will consist of one cohort. Cohort 6 will consist of subjects with impaired hepatic function divided into two groups with some subjects in each of the mild Child-Pugh category (Group 1) and the moderate Child-Pugh category (Group 2).

### Part 1:

Dosing:

Subjects will receive a single ascending dose, under fasting condition, at the following dose levels:

| Cohort | Dose | |
|---|---|---|
| 1 | 27 | mg |
| 2 | 54 | mg |
| 3 | 108 | mg |
| 4 | 162 | mg |

| | |
|---|---|
| 5 | Up to 162 mg |

Each of the cohorts will be dosed sequentially starting with the lowest dose. Subjects enrolled in Cohort 1 can also be enrolled in Cohorts 2, 3, and 4. For each dose cohort, enrollment of subjects with mild or moderate hepatic impairment can be done in parallel. An evaluation of safety and tolerability of the combined mild and moderate hepatic impairment subject data will be done at each dose level before proceeding to the next dose level.

Subjects with severe impairment will be enrolled starting with the lowest dose cohort upon completion of the highest dose tested in subjects with mild or moderate impairment. After review of the safety and tolerability for the first 2 subjects with severe impairment, it will be determined whether or not to complete the dose cohort. An evaluation of safety and tolerability will be done at each dose level for this group.

The drug kinetics in the hepatic impairment subject population will be compared relative to the healthy subject population (Cohort 5).

Blood will be obtained for each cohort at designated times for PK and other analyses (see below). Standard safety assessments will be measured during each treatment period.

### Protocol:

Blood:

Blood from the patients of each cohort will be collected in K₂EDTA tubes. The plasma fraction will be separated by centrifugation and stored frozen until analysis. Blood samples will be collected at the following timepoints: 0 h (prior to the dose), 1.5, 3, 5, 7, 9, 12, 24, 36, 48, and 72 h after the dose.

Safety Assessments/Monitoring

Adverse events (AEs) will be monitored throughout the duration of the study.

To monitor for possible adverse events, sitting blood pressure, heart rate, body temperature, clinical laboratory tests (hematology, chemistry, and urinalysis) and respiration rate will be monitored and physical examination and 12-lead ECG will be conducted during the study.

Statistical Analysis

Statistical analysis will be conducted using statistical methods that are approved for use in FDA clinical trials. Pharmacokinetic analysis will be performed using Phoenix® WinNonlin®, which is validated for bioequivalence/bioavailability studies by inVentiv. Inferential statistical analyses will be performed using SAS® according to FDA guidelines.

The following pharmacokinetic parameters will be calculated by standard non-compartmental methods for nalbuphine and metabolites (if required).
1) AUC₀₋ₜ: area under the concentration-time curve from time zero to the last non-zero concentration;
2) AUC_{0-inf}: area under the concentration-time curve from time zero to infinity (extrapolated);
3) Cₘₐₓ: maximum observed concentration;
4) Tₘₐₓ: time of observed Cₘₐₓ;
5) T_{1/2el}: elimination half-life;
6) Residual area: calculated as 100*(1−AUC₀₋ₜ/AUC_{0-inf});
7) Kₑₗ: elimination rate constant;
8) Cl/F: apparent total body clearance of the drug from plasma; and
9) Vd/F: apparent volume of distribution, calculated as Dose/(Kₑₗ×AUC_{0-inf}).

### Part 2:

In Part 2 of the study (MAD), subjects will receive multiple doses according to a Protocol described in the MAD study described in Example 2. Part 2 of the study may be initiated after Groups 1 and 2 (mild and moderate hepatic impairment) complete Part 1 of the study (SAD) and following satisfactory review of the safety and tolerability data by the Safety Committee. Subjects enrolled in Part 1 can also be enrolled in Part 2.

### Embodiments

1. A method of treating pruritus associated with liver disease comprising orally administering an effective amount of an anti-pruritus agent to a patient in need of such treatment, wherein the anti-pruritus agent is nalbuphine or a pharmaceutically acceptable salt or ester thereof.
2. The method of embodiment 1, wherein the liver disease is selected from the group consisting of cholestatic liver disease, infectious hepatitis, cirrhotic liver disease, drug-induced liver disease, idiopathic portal hypertension, congenital malformations or genetic diseases affecting liver function, sarcoidosis, primary or metastatic neoplasm involvement of the liver and autoimmune hepatitis-cholangitis (Overlap syndrome).
3. A method of treating pruritus associated with obstructive cholestasis secondary to bile duct obstruction due to non-hepatic tissue disease comprising orally administering an effective amount of an anti-pruritus agent to a patient in need of such treatment, wherein the anti-pruritus agent is nalbuphine or a pharmaceutically acceptable salt or ester thereof.
4. The method of embodiment 3, wherein the obstruction results from a condition selected from the group consisting of pancreatic cancer, pancreatitis, congenital or acquired biliary strictures, lymph node obstruction such as from lymphomas or bile duct stones.
5. The method of any one of embodiments 1-4, wherein the pruritus is selected from the group consisting of chronic pruritus, pruritus refractory to treatment with other anti-pruritus agents; pruritus refractory to treatment with bile sequestrants; and pruritus refractory to treatment with rifampicin.
6. The method of any one of embodiments 1-5, wherein the pruritus is chronic pruritus.
7. The method of any one of embodiments 1-6, wherein the pruritus is pruritus refractory to treatment with other anti-pruritus agents.
8. The method of any one of embodiments 1-7, wherein the pruritus is pruritus refractory to treatment with bile sequestrants selected from the group consisting of cholestyramine, colestipol and colesevelam.
9. The method of any one of embodiments 1-8, wherein the pruritus is pruritus refractory to treatment with rifampicin, μ-opioid antagonists, κ-opioid agonists, antidepressants, serotonin antagonists or antihistamines.
10. The method of embodiment 9, wherein the κ-opioid agonist is nalfurafine.
11. The method of embodiment 9, wherein the μ-opioid antagonist is naltrexone.
12. The method of any one of embodiments 1-2 and 5-11, wherein the patient does not have a bile duct obstruction.
13. The method of any one of embodiments 1-2 and 5-12, wherein the liver disease is cholestatic liver disease.
14. The method of embodiment 13, wherein the cholestatic liver disease is selected from primary sclerosing cholangitis and primary biliary cholangitis.
15. The method of any one of embodiments 1-2 and 5-12, wherein the liver disease is infectious hepatitis.
16. The method of embodiment 15, wherein the infectious hepatitis is selected from hepatitis C (HCV) and hepatitis B (HBV).
17. The method of embodiment 16, wherein the HCV is selected from chronic HCV and HCV post sustained virologic response.
18. The method of embodiment 16, wherein the hepatitis B is selected from inactive HBV in a carrier and active HBV infection.
19. The method of any one of embodiments 1-2 and 5-12, wherein the liver disease is cirrhotic liver disease.
20. The method of embodiment 19, wherein the cirrhotic liver disease is selected from alcoholic liver disease, autoimmune hepatitis, and non-alcoholic fatty liver disease.
21. The method of any one of embodiments 1-2 and 5-12, wherein the liver disease is selected from drug-induced liver disease, idiopathic portal hypertension, congenital malformations or genetic diseases affecting liver function, sarcoidosis, primary or metastatic neoplasm involvement of the liver and autoimmune hepatitis-cholangitis (Overlap syndrome).
22. The method of any one of embodiments 1-21, wherein patient's serum levels of endogenous opioids are elevated compared to normal serum levels.
23. The method of any one of embodiments 1-22, wherein about 14 mg of the Equivalent Amount of Nalbuphine Free Base is administered once a day.
24. The method of any one of embodiments 1-22, wherein about 14 mg of the Equivalent Amount of Nalbuphine Free Base is administered twice a day.
25. The method of any one of embodiments 1-22, wherein about 27 mg of the Equivalent Amount of Nalbuphine Free Base is administered once a day.
26. The method of any one of embodiments 1-22, wherein about 27 mg of the Equivalent Amount of Nalbuphine Free Base is administered twice a day.
27. The method of any one of embodiments 1-22, wherein about 54 mg of the Equivalent Amount of Nalbuphine Free Base is administered once a day.
28. The method of any one of embodiments 1-22, wherein about 54 mg of the Equivalent Amount of Nalbuphine Free Base is administered twice a day.
29. The method of any one of embodiments 1-22, wherein about 81 mg of the Equivalent Amount of Nalbuphine Free Base is administered once a day.
30. The method of any one of embodiments 1-22, wherein about 81 mg of the Equivalent Amount of Nalbuphine Free Base is administered twice a day.
31. The method of any one of embodiments 1-22, wherein about 108 mg of the Equivalent Amount of Nalbuphine Free Base is administered once a day.
32. The method of any one of embodiments 1-22, wherein about 108 mg of the Equivalent Amount of Nalbuphine Free Base is administered twice a day.
33. The method of any one of embodiments 1-22, wherein about 162 mg of the Equivalent Amount of Nalbuphine Free Base is administered once a day.
34. The method of any one of embodiments 1-22, wherein about 162 mg of the Equivalent Amount of Nalbuphine Free Base thereof is administered twice a day.
35. The method of any one of embodiments 1-22, wherein about 324 mg of the Equivalent Amount of Nalbuphine Free Base is administered once a day.
36. The method of any one of embodiments 1-22, wherein about 15 mg of nalbuphine or a pharmaceutically acceptable salt or ester thereof is administered once a day.
37. The method of any one of embodiments 1-22, wherein about 15 mg of nalbuphine or a pharmaceutically acceptable salt or ester thereof is administered twice a day.
38. The method of any one of embodiments 1-22, wherein about 30 mg of nalbuphine or a pharmaceutically acceptable salt or ester thereof is administered once a day.
39. The method of any one of embodiments 1-22, wherein about 30 mg of nalbuphine or a pharmaceutically acceptable salt or ester thereof is administered twice a day.
40. The method of any one of embodiments 1-22, wherein about 60 mg of nalbuphine or a pharmaceutically acceptable salt or ester thereof is administered once a day.
41. The method of any one of embodiments 1-22, wherein about 60 mg of nalbuphine or a pharmaceutically acceptable salt or ester thereof is administered twice a day.
42. The method of any one of embodiments 1-22, wherein about 90 mg of nalbuphine or a pharmaceutically acceptable salt or ester thereof is administered once a day.
43. The method of any one of embodiments 1-22, wherein about 90 mg of nalbuphine or a pharmaceutically acceptable salt or ester thereof is administered twice a day.
44. The method of any one of embodiments 1-22, wherein about 120 mg of nalbuphine or a pharmaceutically acceptable salt or ester thereof is administered once a day.
45. The method of any one of embodiments 1-22, wherein about 120 mg of nalbuphine or a pharmaceutically acceptable salt or ester thereof is administered twice a day.
46. The method of any one of embodiments 1-22, wherein about 180 mg of nalbuphine or a pharmaceutically acceptable salt or ester thereof is administered once a day.
47. The method of any one of embodiments 1-22, wherein about 180 mg of nalbuphine or a pharmaceutically acceptable salt or ester thereof is administered twice a day.
48. The method of any one of embodiments 1-22, wherein about 360 mg of nalbuphine or a pharmaceutically acceptable salt or ester thereof is administered once a day.
49. The method of any one of embodiments 1-48, wherein said administering is for about 8 weeks, about 10 weeks, about 12 weeks, about 24 weeks or about 50 weeks.
50. The method of any one of embodiments 1-49, further comprising titrating the dose of the anti-pruritus agent for at least one week until a steady state is achieved in the patient.
51. The method of any one of embodiments 1-49, further comprising titrating the dose of the anti-pruritus agent for about 2 weeks until a steady state is achieved in the patient.
52. The method of any one of embodiments 1-49, further comprising titrating the dose of the anti-pruritus agent for about 7 to 30 days until a steady state is achieved in the patient.
53. The method of any one of embodiments 1-49, further comprising titrating the dose of the anti-pruritus agent for about 14 to 20 days until a steady state is achieved in the patient.
54. The method of embodiment 51, wherein said titrating comprises administering ascending doses of the anti-pruritus agent until a steady state is achieved in the patient.
55. The method of embodiment 51, wherein said titrating comprises administering ascending doses of the anti-pruritus agent until an effective amount of 15 mg, 30 mg, 60 mg, 90 mg, 120 mg, 180 mg, 240 mg or 360 mg is achieved in the patient.
56. The method of embodiment 51, wherein said titrating comprises administering ascending doses of the Equivalent Amount of Nalbuphine Free Base until an effective amount of 14 mg, 27 mg, 54 mg, 81 mg, 108 mg, 162 mg, 216 mg or 324 mg is achieved in the patient.
57. The method of embodiment 51, wherein said titrating further comprises administering an initial dose of about 30 mg once or twice a day.
58. The method of embodiment 50, wherein said titrating comprises administering the anti-pruritus agent in increments ranging from about 15 mg to about 60 mg.
59. The method of embodiment 50, wherein said titrating further comprises administering an initial dose of about 27 mg of the Equivalent Amount of Nalbuphine Free Base once or twice a day.
60. The method of embodiment 50, wherein said titrating comprises administering the anti-pruritus agent in increments ranging from about of the Equivalent Amount of Nalbuphine Free Base 14 mg to about 54 mg.
61. The method of any one of embodiments 50, wherein said administering comprises twice a day administration with an AM dosage and a PM dosage, wherein the PM dosage is higher than or the same as the AM dosage.
62. The method of any one of embodiments 1-61, wherein after said treating the patient experiences a substantial reduction in itch compared to prior to said treating.
63. The method of any one of embodiments 1-62, wherein after said treating the patient experiences a reduction of itch that is characterized by an at least two point decline in worst itching intensity Numerical Rating Scale (NRS) value.
64. The method of embodiment 63, wherein the reduction of itch is an at least three point decline in worst itching intensity NRS value.
65. The method of embodiment 63, wherein the reduction of itch is an at least four point decline in worst itching intensity NRS value.
66. The method of any one of embodiments 1-65, wherein after said treating the patient experiences a reduction of itch that is characterized by an at least two point decline in average itching intensity Numerical Rating Scale (NRS) value.
67. The method of embodiment 66, wherein the reduction of itch is an at least three point decline in average itching intensity NRS value.
68. The method of embodiment 66, wherein the reduction of itch is an at least four point decline in average itching intensity NRS value.
69. The method of any one of embodiments 1-68, wherein after said treating the patient experiences a reduction of itch that is characterized by at least about 10 mm change in visual analogue scale worst itch or average itch (VAS) value (using VAS scale ranging from “no itch at VAS=0 to “worst possible itch” at VAS=100 mm).
70. The method of embodiment 69, wherein after said treating the patient experiences a reduction of itch that is characterized by at least about 20 mm change in worst itch or average itch VAS value (using VAS scale ranging from “no itch at VAS=0 to “worst possible itch” at VAS=100 mm).
71. The method of embodiment 69, wherein after said treating the patient experiences a reduction of itch that is characterized by at least about change 30 mm in worst itch or average itch VAS value (using VAS scale ranging from “no itch at VAS=0 to “worst possible itch” at VAS=100 mm).
72. The method of any one of embodiments 1-71, wherein the nalbuphine or a pharmaceutically acceptable salt or ester thereof is administered in conjunction with one or more anti-pruritic agents.
73. The method of embodiment 72, wherein the one or more anti-pruritic agents is selected from the group consisting of antihistamines, antidepressants, serotonin antagonists, anti-inflammatory corticosteroids, topical anti-infectives and antifungals, antibacterials, antivirals, cytotoxic agents, and counterirritants/analgesics.
74. The method of any one of embodiments 1-73, wherein the anti-pruritus agent is nalbuphine hydrochloride.
75. The method of any one of embodiments 1-74, wherein the anti-pruritus agent is in the form of an extended release oral dosage form.
76. The method of any one of embodiments 1-75, wherein the anti-pruritus agent is administered in a formulation comprising nalbuphine hydrochloride, mannitol, hydroxypropyl cellulose, locust bean gum, xanthan gum, calcium sulfate dihydrate, fumaric acid and magnesium stearate.
77. The method of any one of embodiments 1-76, wherein the patient is a pediatric patient.
78. The method of any one of embodiments 1-76, wherein the patient is a geriatric patient.

What is claimed is:

1. A method of treating pruritus associated with liver disease comprising orally administering an effective amount of nalbuphine or a pharmaceutically acceptable salt or ester thereof to a patient in need of such treatment,
wherein the liver disease is selected from the group consisting of cholestatic liver disease, infectious hepatitis, cirrhotic liver disease, drug-induced liver disease, idiopathic portal hypertension, congenital malformations or genetic diseases affecting liver function, sarcoidosis, primary or metastatic neoplasm involvement of the liver and autoimmune hepatitis-cholangitis (Overlap syndrome).

2. The method of claim 1, wherein the pruritus is selected from the group consisting of chronic pruritus, pruritus refractory to treatment with other anti-pruritus agents; pruritus refractory to treatment with bile sequestrants; and pruritus refractory to treatment with rifampicin.

3. The method of claim 1, wherein the pruritus is chronic pruritus.

4. The method of claim 1, wherein the pruritus is pruritus refractory to treatment with other anti-pruritus agents.

5. The method of claim 1, wherein the pruritus is pruritus refractory to treatment with bile sequestrants selected from the group consisting of cholestyramine, colestipol and colesevelam.

6. The method of claim 1 wherein the pruritus is pruritus refractory to treatment with rifampicin, μ-opioid antagonists, κ-opioid agonists, antidepressants, serotonin antagonists or antihistamines.

7. The method of claim 6, wherein the κ-opioid agonist is nalfurafine.

8. The method of claim 6, wherein the μ-opioid antagonist is naltrexone.

9. The method of claim 1, wherein the patient does not have a bile duct obstruction.

10. The method of claim 1, wherein the liver disease is cholestatic liver disease.

11. The method of claim 10, wherein the cholestatic liver disease is selected from primary sclerosing cholangitis and primary biliary cholangitis.

12. The method of claim 1, wherein the liver disease is infectious hepatitis.

13. The method of claim 12, wherein the infectious hepatitis is selected from hepatitis C (HCV) and hepatitis B (HBV).

14. The method of claim 13, wherein the HCV is selected from chronic HCV and HCV post sustained virologic response.

15. The method of claim 13, wherein the hepatitis B is selected from inactive HBV in a carrier and active HBV infection.

16. The method of claim 1, wherein the liver disease is cirrhotic liver disease.

17. The method of claim 14, wherein the cirrhotic liver disease is selected from alcoholic liver disease, autoimmune hepatitis, and non-alcoholic fatty liver disease.

18. The method of claim 1, wherein the liver disease is selected from drug-induced liver disease, idiopathic portal hypertension, congenital malformations or genetic diseases affecting liver function, sarcoidosis, primary or metastatic neoplasm involvement of the liver and autoimmune hepatitis-cholangitis (Overlap syndrome).

19. The method of claim 1, wherein patient's serum levels of endogenous opioids are elevated compared to normal serum levels.

20. The method of claim 1, wherein about 15 mg of nalbuphine or a pharmaceutically acceptable salt or ester thereof is administered once a day.

21. The method of claim 1, wherein about 15 mg of nalbuphine or a pharmaceutically acceptable salt or ester thereof is administered twice a day.

22. The method of claim 1, wherein about 30 mg of nalbuphine or a pharmaceutically acceptable salt or ester thereof is administered once a day.

23. The method of claim 1, wherein about 30 mg of nalbuphine or a pharmaceutically acceptable salt or ester thereof is administered twice a day.

24. The method of claim 1, wherein about 60 mg of nalbuphine or a pharmaceutically acceptable salt or ester thereof is administered once a day.

25. The method of claim 1, wherein about 60 mg of nalbuphine or a pharmaceutically acceptable salt or ester thereof is administered twice a day.

26. The method of claim 1, wherein about 90 mg of nalbuphine or a pharmaceutically acceptable salt or ester thereof is administered once a day.

27. The method of claim 1, wherein about 90 mg of nalbuphine or a pharmaceutically acceptable salt or ester thereof is administered twice a day.

28. The method of claim 1, wherein about 120 mg of nalbuphine or a pharmaceutically acceptable salt or ester thereof is administered once a day.

29. The method of claim 1, wherein about 120 mg of nalbuphine or a pharmaceutically acceptable salt or ester thereof is administered twice a day.

30. The method of claim 1, wherein about 180 mg of nalbuphine or a pharmaceutically acceptable salt or ester thereof is administered once a day.

31. The method of claim 1, wherein about 180 mg of nalbuphine or a pharmaceutically acceptable salt or ester thereof is administered twice a day.

32. The method of claim 1, wherein about 360 mg of nalbuphine or a pharmaceutically acceptable salt or ester thereof is administered once a day.

33. The method of claim 1, wherein said administering is for about 8 weeks, about 10 weeks, about 12 weeks, about 24 weeks or about 50 weeks.

34. The method of claim 1, further comprising titrating the dose of the anti-pruritus agent for at least one week until a steady state is achieved in the patient.

35. The method of claim 1, further comprising titrating the dose of the anti-pruritus agent for about 2 weeks until a steady state is achieved in the patient.

36. The method of claim 1, further comprising titrating the dose of the anti-pruritus agent for about 7 to 30 days until a steady state is achieved in the patient.

37. The method of claim 1, further comprising titrating the dose of the anti-pruritus agent for about 14 to 20 days until a steady state is achieved in the patient.

38. The method of claim 34, wherein said titrating comprises administering ascending doses of the anti-pruritus agent until a steady state is achieved in the patient.

39. The method of claim 34, wherein said titrating comprises administering ascending doses of the anti-pruritus agent until an effective amount of 15 mg, 30 mg, 60 mg, 90 mg, 120 mg, 180 mg, 240 mg or 360 mg is achieved in the patient.

40. The method of claim 34, wherein said titrating further comprises administering an initial dose of about 30 mg once or twice a day.

41. The method of claim 34, wherein said titrating comprises administering the anti-pruritus agent in increments ranging from about 15 mg to about 60 mg.

42. The method of claim 1, wherein after said treating the patient experiences a substantial reduction in itch compared to prior to said treating.

43. The method of claim 1, wherein after said treating the patient experiences a reduction of itch that is characterized by an at least two point decline in worst itching intensity Numerical Rating Scale (NRS) value.

44. The method of claim 43, wherein the reduction of itch is an at least three point decline in worst itching intensity NRS value.

45. The method of claim 43, wherein the reduction of itch is an at least four point decline in worst itching intensity NRS value.

46. The method of claim 1, wherein after said treating the patient experiences a reduction of itch that is characterized by an at least two point decline in average itching intensity NRS value.

47. The method of claim 46, wherein the reduction of itch is an at least three point decline in average itching intensity NRS value.

48. The method of claim 46, wherein the reduction of itch is an at least four point decline in average itching intensity NRS value.

49. The method of claim 1, wherein after said treating the patient experiences a reduction of itch that is characterized by at least about 10 mm change in visual analogue scale worst itch or average itch (VAS) value (using VAS scale ranging from “no itch at VAS=0 to “worst possible itch” at VAS=100 mm).

50. The method of claim 49, wherein after said treating the patient experiences a reduction of itch that is characterized by at least about 20 mm change in worst itch or average itch VAS value (using VAS scale ranging from “no itch at VAS=0 to “worst possible itch” at VAS=100 mm).

51. The method of claim 49, wherein after said treating the patient experiences a reduction of itch that is characterized by at least about change 30 mm in worst itch or average itch VAS value (using VAS scale ranging from “no itch at VAS=0 to “worst possible itch” at VAS=100 mm).

52. The method of claim 1, wherein the nalbuphine or a pharmaceutically acceptable salt or ester thereof is administered in conjunction with one or more anti-pruritic agents.

53. The method of claim 52, wherein the one or more anti-pruritic agents is selected from the group consisting of antihistamines, antidepressants, serotonin antagonists, anti-inflammatory corticosteroids, topical anti-infectives and antifungals, antibacterials, antivirals, cytotoxic agents, and counterirritants/analgesics.

54. The method of claim 1, wherein the nalbuphine or a pharmaceutically acceptable salt or ester thereof is nalbuphine hydrochloride.

55. The method of claim 1, wherein the nalbuphine or a pharmaceutically acceptable salt or ester thereof is in the form of an extended release oral dosage form.

56. The method of claim 1, wherein the nalbuphine or a pharmaceutically acceptable salt or ester thereof is administered in a formulation comprising nalbuphine hydrochloride, mannitol, hydroxypropyl cellulose, locust bean gum, xanthan gum, calcium sulfate dihydrate, fumaric acid and magnesium stearate.
